(19) United States
(12) Patent Application Publication
(10) Pub. No.: US 2026/0028566 A1
(43) Pub. Date: Jan. 29, 2026

(54) TUBULAR STRUCTURES, METHODS, AND APPARATUSES FOR MAKING AND USING THE SAME

(71) Applicant: THE TEXAS A&M UNIVERSITY SYSTEM, College Station, TX (US)

(72) Inventors: Tracy M. CLEMENT, College Station, TX (US); Jacob Thomas CABLER, College Station, TX (US)

(21) Appl. No.: 19/104,311

(22) PCT Filed: Aug. 25, 2023
(86) PCT No.: PCT/US2023/031173

§ 371 Date: Feb. 17, 2025

Related U.S. Application Data
(60) Provisional application No. 63/401,225, filed on Aug. 26, 2022.

(51) Int. Cl. C12M 1/12

(52) U.S. Cl. CPC C12M 23/06; C12M 25/14

(57) ABSTRACT

Tubular structures, apparatuses, and methods for making tubular structures are disclosed herein. Walls of the tubular structures can include a hydrogel. A luminal material extends through the tubular wall. A cell population can live in the luminal material, the tubular wall, and/or on an outer surface of a tubular structure. Apparatuses for making tubular structures can include one or more inner extrusion nozzles removably positioned coaxially within one or more outer extrusion nozzles. The outer extrusion nozzle can be removably or permanently positioned within a housing of the apparatus, positioned above a column-shaped container. Methods of making a tubular structure can include coextruding a wall solution and a luminal solution into the column-shaped container and polymerizing the wall solution into a tubular structure. In some aspects, a light source can be configured to illuminate a light-permeable wall of the container or the nozzles for the purpose of polymerizing the wall solution.

### RELATED APPLICATIONS

This application claims the benefit of U.S. Provisional Application No. 63/401,225, filed Aug. 26, 2022, which is hereby incorporated by reference in its entirety.

### FIELD

This disclosure relates to tissue engineering, and more particularly to the engineering of small tubular biological structures.

### BACKGROUND

Microphysiological systems, such as organ-on-chip systems, aim to replicate the physiological 3D architecture, morphology, and micro-anatomy of tissues such that a cultured tissue's functional states can better replicate its physiological counterpart. The design of such systems are composed of a mix of tissue engineering and cell and/or tissue culturing techniques. It is understood that cellular arrangements, including cellular proximities and spatial arrangements, are key to proper tissue function in vivo. In addition, fluid dynamics are a key characteristic of many tissues where interstitial flow and flow in tubular tissues, such as vasculature or lymphatics, contribute to tissue and organ maintenance and function. Additionally, in other tubular tissues such as renal tubules, oviducts, seminiferous tubules, etc., not only are the cellular composition and arrangement important, but the fluid dynamics in the luminal and extraluminal compartments are also important aspects of tissue function which can be regulated or influenced by, within, or outside the target tissue.

Organ-on-chip microphysiological systems can allow control over their hydrostatic and hydrodynamic variables, but such systems are currently limited in the 3D arrangements that can be produced, particularly for tubular tissues. This limits control of hydrostatic and hydrodynamic variables that is possible between luminal and extraluminal compartments. In vitro tubular tissues can be formed by microchannels in devices, through additive techniques, or through extrusion, each of which has limitations. Microchannels in devices limit compartmentalization and compartment access, as well as permeability through the matrix in which the channel resides. Additive techniques limit the minimum producible size of tube lumens and wall thicknesses. Extrusion requires mixed materials to generate tubular structures through various polymerization techniques and can be limited by size restriction, necessary use of undesirable additives for polymerization, excessive stress such as sheer on cell populations, and/or lack of consistent geometry and patent lumen production. These limitations prevent these systems from providing an accurate representation of physiological tubular tissues. Thus, there is a need to form small, hollow; tubular structures having radially symmetrical walls of reliable, uniform thickness in a desired range with desired porosity and permeability. These needs and others are at least partially satisfied by the present disclosure.

### SUMMARY

The tubular structures and organoids disclosed herein comprise a tubular wall that includes a hydrogel material. A lumen extends through the tubular wall and comprises a luminal material. The tubular wall has a first end, a second end, and a radial thickness t ranging from about 1 microns to about 200 microns. In some aspects, a standard error of the radial thickness can be relatively low, for example, from about 5% to about 20% of the radial thickness. A cell population can live in the luminal material, one or more layers of the tubular wall, and/or on an outer surface of the tubular wall.

The hydrogel material of the tubular wall can be free of divalent cations. In some aspects, the hydrogel material is free of alginate. The hydrogel material can include gelatin or gelatin methacryloyl (Gelma). In some aspects, the hydrogel material includes decellularized extracellular matrix. In some aspects, other photoactivatable material such as PEGDA or non-photoactivatable materials such as PEG, decellularized extracellular matrix, modified signaling peptides, additional hydrogels, or additives may be included in the tubular wall materials in one or more layers to allow for alterations in material properties such as strength, stiffness, porosity, surface texture, cell adhesiveness, and/or changes in cellular affinity/signaling such as through tissue specific matrix or signaling factors. The materials used in each layer can be low or high viscosity and can be density matched to reduce chaotic flow.

The tubular wall has an outer diameter measured between outer surfaces of the tubular wall. The outer diameter of the tubular wall can, in some aspects, range from about 5 microns to about 300 microns. The standard of error of the outer diameter can be from about 3% to about 10% of the outer diameter. The tubular wall has a luminal diameter defined by inner surfaces of the tubular wall. The luminal diameter can range from about 1 micron to about 200 microns. In some aspects, the wall thickness is about 1 micron to about 200 microns and may be composed of one or more radial layers comprising the total thickness. In some aspects, the standard error of the luminal diameter is less than about 8%. In some aspects the thickness of individual wall layers can be about 0. 1 microns to about 200 microns.

In some aspects, the cell population includes cells that are derived from, or mimic, the cells of a reproductive organ. For example, in some aspects, the cell population comprises testicular cells. The organoid can mimic a seminiferous tubule. In some aspects, the tubular organoid produces haploid germ cells. In some aspects, the cell population can include epithelial cells. In some aspects, the cell population comprises smooth muscle cells. In some examples, a first cell population lives in the tubular wall and a second cell population lives in the luminal material. Additional layers of the tubular wall component can contain different cell populations as well. Alternatively or in addition, a cell population can live on an outer surface of the tubular material.

Apparatuses for coextruding tubular structures are also disclosed herein. Apparatuses include a housing having an upper compartment and a lower compartment. In some aspects, the upper compartment includes opaque walls. In some aspects the compartments are separated only by a light shield blocking light-sensitive materials. In some aspects, the lower compartment includes light permeable walls.

The apparatuses further include one or more outer extrusion nozzles, which can be nested within each other, and one or more inner extrusion nozzles, which can also be nested within each other and within the outer extrusion nozzle(s). The outer extrusion nozzle(s) can be removably or permanently positioned within the housing and can extend from the upper compartment into the lower compartment. The inner extrusion nozzle(s) can be removably or permanently positioned coaxially within the outer extrusion nozzle(s). The apparatuses further include a column-shaped container with a light-permeable wall positioned beneath the outer and inner extrusion nozzles. In some aspects, the container holds an aqueous solution such as buffer or media. A light source can be positioned inside the lower compartment and configured to illuminate the light-permeable wall of the container. In some aspects, a light source can be positioned outside the lower compartment and configured to illuminate the light-permeable walls of the lower compartment and the container. In some aspects, one or more of the extrusion nozzles include a light-permeable material such that crosslinking can be initiated within the extrusion nozzle.

In some aspects, the outer nozzle tapers to an outer nozzle tip and comprises an interior pore defined by an inner surface of the outer nozzle. The interior pore can have a diameter of less than 400 microns at an outer nozzle tip. In some aspects, the inner extrusion nozzle ends at an inner nozzle tip that is positioned within the interior pore of the outer extrusion nozzle. In some aspects, an outer diameter of the outer nozzle tip is smaller than an outer diameter of the inner nozzle tip. In some aspects there is more than one inner nozzle or more than one inner nozzle tip. When more than two layers are generated, the outer nozzle can contain the wall material, or a non-polymerizing material to aid in reduction of the tubular radial thickness. In some aspects the inner layers can form one or more layers of a tubular wall. In some aspects the inner most nozzle or tip contains non-polymerizing material to allow for a patent lumen in the tube.

Some aspects include a pump fluidically coupled to an interior pore of the outer extrusion nozzle. Some aspects include a pump fluidically coupled to an interior pore of the inner extrusion nozzle. Some aspects include different pumps fluidically coupled to interior pores of different nested outer extrusion nozzles. Likewise, some aspects include different pumps fluidically coupled to the interior pores of different nested inner extrusion nozzles. The rate of fluid flow for materials in each layer of the tube can be controlled such that the ratios of flow contribute to the thickness of each layer and compartment. In some aspects the flow rates for material extruded in each layer is calculated based on the cross-sectional area ratios of the extrusion tip to match velocities of the extruded materials. In some aspects the extrusion tips are optimized for the desired layer thicknesses by optimizing the cross-sectional areas at the point of each layer extruding into the next, such that combined with proportional flow rates, the velocity of material being extruded at this point of extrusion for each layer into the next is matched.

In some aspects, the light source includes an elongated array aligned with the container. In some aspects there is a single point source of light. The light source can emit at wavelengths that optimize polymerization of the photoactivatable material and cell sensitivities and can include UVA, violet, or blue light in some aspects. The light source intensity can be controllable. In some aspects, the apparatuses include an environmental regulation system configured to control environmental conditions within the housing. The environmental regulation system can include at least one of a light ballast, a light regulator, a heating component, humidity control, and a control panel.

Methods of making a tubular structure or a tubular organoid are disclosed herein. The methods include steps of coextruding at least one wall solution through at least one outer nozzle, coextruding a luminal solution through at least one inner nozzle positioned inside the outer nozzle, directing the wall solution and the luminal solution into a column-shaped container filled with extrusion buffer, and polymerizing the wall solution with light into a tubular structure. In some methods, coextruding the wall solution includes extruding a wall thickness of 1 microns to 200 microns. Some methods include extruding multiple radial wall layers, wherein a radial wall layer comprises a thickness of from 0.1 microns to 200 microns. In some methods, polymerizing the wall solution results in a tubular structure or tubular organoid comprising a length of at least 1 meter between a first end and a second end.

In some aspects of the methods, the step of directing the wall solution and the luminal solution into a column-shaped container can include coextruding the wall solution and the luminal solution directly into the extrusion buffer. In some methods, coextruding the wall solution occurs at a flow rate of from 1 microliter/minute to 100 microliters/minute. In some methods, coextruding the luminal solution occurs at a flow rate of from 1 microliter/minute to 100 microliters/minute. In some methods, the wall solution, the luminal solution, or both carry a cell population.

Some aspects of the methods of making a tubular structure or a tubular organoid include a step of controlling an environmental condition to optimize a polymerization rate. The step of polymerizing the wall solution can take place within the outer extrusion nozzle, within the container, and/or as the wall solution sinks within the media or buffer of the container.

In some methods, the step of polymerizing the wall solution can include photo crosslinking the wall solution as it sinks within the extrusion buffer, for example, by illuminating the wall solution with UVA, violet, or blue light through a light-permeable wall of the container. In some methods, greater illumination is applied near the top of the container than the bottom of the container. In some methods, there is a single point source of light. In some methods the outer most layer is a non-photoactivatable material, and the light may be directed to the final portion of the outer extrusion tip before final extrusion.

Tubular organoids may also be formed by extraction of extracellular matrix of tubular tissues where the 3D integrity of tubular structure is maintained. For example, decellularization of testis tissue such as seminiferous tubules which can then provide the cell-free tubular constructs to be used as the base for cell seeding to generate tubular organoids. In some cases, the extracellular matrix constructs may be generated by treating tissues with freeze-thaw cycles, DNase, detergents, or other processes to generate in-tact extracellular matrix from tissues. Thes may be extracted from any tubular structure in any species, such as seminiferous epithelium, lymphatics, or kidney tissues in rodents, bovine, porcine, or even invertebrate species.

Tubular organoids may be produced by templating where the lumen of the tubular construct is produced by templating in the hydrogel material. In this case the material may be photopolymerizable hydrogel such as GELMA or PEGDA. The templating may be by dipping a core filament into the material to be cured or may be a filament in a cylindrical or block mold for example. These could be aspects of a tissue chip design where a luminal compartment is left in a tubular structure, or in a structure with a media perusable hydrogel. The gel thickness may be a thickness appropriate to replicate the hydrodynamic and hydrostatic requirements such as hydrostatic pressure, hydrostatic pressure differentials, fluid flow, shear, diffusion of media and components contained within, etc. for organoid development and function.

Tubular organoids may be generated by bioprinting such as by using additive manufacturing techniques to build photopolymerizable materials into the small tubular structures needed to build tubular organoids. Photopolymerizable materials may include hydrogels such as GELMA or PEGDA or other photocuring agents such as ceramics to form the basis of the tubular organoid constructs to be used for cellular seeding. Additive manufacturing may include light based layered bed printing in which designs leave a lumen unpolymerized, or additive polymerization may be completed around a core material that can later be removed to leave the luminal compartment.

Methods of using a tubular organoid can include forming a tubular organoid, such as an organoid that mimics a testicular structure, via the methods of making described above. The methods of using a tubular organoid can further include contacting the tubular organoid with an agent or condition and monitoring the effects of the agent or condition on a measure of organoid function (such as testicular function).

In some methods, the step of contacting the tubular organoid with the agent or condition includes introducing the agent or condition into the tubular organoid. In some methods, the agent or condition can be a contraceptive agent, a potential toxin, a biological factor, an electrical stimulus, and/or a mechanical stimulus.

A step of forming a tubular organoid that mimics a testicular structure can include subjecting the tubular organoid to a testicular maturation protocol. This may include distinct phases of addition or maturation for one or more cell type to be incorporated into the organoids.

In some methods, the testicular structure is the seminiferous epithelium. In some methods, the measure of testicular function is a quantity of maturing male germ cells formed by the tubular organoid. In some methods, the measure of testicular function is a tortuosity of the tubular organoid. In some methods, the tubular organoid includes a non-germ cell population.

Methods of preserving fertility are disclosed herein, the methods including steps of harvesting one or more cells from a subject, making a tubular structure via the methods described above, subjecting the tubular structure to a maturation protocol that induces maturation of the tubular structure into a tubular organoid, preserving the tubular organoid, and harvesting a cellular material from the tubular organoid. In some aspects, the method includes mixing the one or more cells from the subject with the wall solution, the luminal solution, or both prior to making the tubular structure. In some aspects, the step of subjecting the tubular structure to a maturation protocol includes seeding the one or more cells from the subject into the tubular structure. In some aspects, subjecting the tubular structure to a maturation protocol includes maintaining the tubular organoid in a tissue chip or bioreactor. In some aspects of the methods of preserving fertility, the maturation protocol causes the tubular organoid to mimic a testicular structure. In some methods, the testicular structure is a seminiferous epithelium. In some methods, the germ cell is haploid.

In some methods the cellular composition of the organoids may include cells from all the same species, or combine cells from different species. In some methods the cells may be from rodents, humans, bovine, or porcine for example. In some methods some of the cell populations may be derived from species known to have regenerative properties such as spiny mice or axolotl. For example, human germ cells harvested from patients or derived from stem cell or induced pluripotent germ cells or other human derived sources may be combined with cell types from other species to generate human models of spermatogenesis. These organoids may be used for clinical or research purposes, or for screening of compounds such as toxicants or male contraceptives. Similarly, organoids for other tubular tissue types may be generated using combinations of cell-types derived from the same or different species.

In some aspects, the techniques described herein relate to a tissue bioreactor including: at least one extra-luminal compartment including a first fluid and a compartment wall; a first cannula extending at least partially into the extra-luminal compartment; a second cannula extending at least partially into the extra-luminal compartment at a position separate from the first cannula; and a tubular structure at least partially submerged in the first fluid and fluidically coupling the first cannula to the second cannula, the tubular structure defining a luminal compartment. The extra-luminal compartment can be fluidically coupled to a first fluid source and the luminal compartment is fluidically coupled to a second fluid source, and the bioreactor can be configured to maintain distinct conditions within the luminal compartment and the extra-luminal compartment.

In some aspects, the techniques described herein relate to a tissue chip including: at least one extra-luminal compartment including a compartment wall, the compartment wall including an inlet and an outlet; a first cannula extending at least partially through the compartment wall at a position separate from the inlet; and a second cannula extending at least partially through the compartment wall at a position separate from the inlet and the first cannula; wherein the first cannula and second cannula are configured to be fluidically connected to each other via a tubular structure located at least partially within the extra-luminal compartment. The inlet can be configured for fluidic coupling to a first fluid source and the first cannula can be configured for fluidic coupling to a second fluid source. The tissue chip can be configured to prevent mixing of fluids between the extra-luminal compartment and a luminal compartment (the luminal compartment comprising a lumen of a tubular structure extending between the first cannula and the second cannula).

In some aspects the tissue chip can include a base defining the extra-luminal compartment and a releasable cover configured to releasably seal to the base.

In some aspects, the tissue chip includes the tubular structure, and the tubular structure includes: a tubular wall including a hydrogel material, the tubular wall including a first end, a second end, and a radial thickness ranging from 1 micron to 200 microns; a lumen extending through the tubular wall and including a luminal diameter defined by inner surfaces of the tubular wall; and a luminal material within the lumen.

In some aspects, the techniques described herein relate to methods of using a tissue chip, wherein the methods can include: fluidically coupling an inlet of the tissue chip to a first fluid source; fluidically coupling a first end portion of a tubular structure to a first cannula, the first cannula extending at least partially into an extra-luminal compartment of the tissue chip; fluidically coupling a second end portion of the tubular structure to a second cannula, the second cannula extending at least partially into the extra-luminal compartment of the tissue chip; fluidically coupling the first cannula to a second fluid source; initiating flow from the first fluid source through the inlet and into the extra-luminal compartment; initiating flow from the second fluid source into a luminal compartment of the tubular structure; and maintaining patency of a wall of the tubular structure such that the extra-luminal compartment remains separate from the luminal compartment.

In some aspects, the methods of using a tissue chip, further include suspending the tubular structure within the extra-luminal compartment. In some aspects, the tubular structure is pre-seeded with cells. Some aspects of the methods include maintaining a cell culture media factor at a different concentration in the extra-luminal compartment than in the luminal compartment.

In some aspects, the methods of using a tissue chip further include regulating one or more hydrostatic or hydrodynamic properties of the luminal compartment, the extra luminal compartment, or both. The one or more hydrostatic or hydrodynamic properties can include a hydrostatic pressure, a hydrostatic pressure differential between the extra-luminal compartment and the luminal compartment, a fluid flow rate, a shear stress, and a media exchange rate. In some aspects of the methods of using a tissue chip, regulating one or more hydrostatic or hydrodynamic properties exerts temporal control over an addition of a factor to a cell culture media. In some aspects, regulating the one or more hydrostatic or hydrodynamic properties includes using one or more of the following; one or more pumps, a gravity feed, a rocking device, a rotating device, fluid restricting channels, and buffer/media wells.

Some aspects of the methods of using a tissue chip further include releasably sealing a cover to a base of the tissue chip to close the extra-luminal compartment. Some aspects further include releasing the cover to access the extra-luminal compartment.

In some aspects, the methods of using a tissue chip further include sampling media from the extra-luminal compartment, the luminal compartment, or both. Sampling media can include analyzing sloughed cells and, in some aspects, analyzing sloughed cells includes testing for germ cell maturity. In some aspects, sampling media includes analyzing cellular materials, wherein cellular materials can include metabolites, small molecules, proteins, nucleic acids, signaling factors, germ cells, and vesicles, for example.

In some aspects, the methods of using a tissue chip further include accessing the extra-luminal compartment, the luminal compartment, or both to conduct measurements of tissue barrier integrity. For example, the compartments can

### DESCRIPTION OF DRAWINGS

The device is explained in even greater detail in the following drawings. The drawings are merely exemplary and certain features may be used singularly or in combination with other features. The drawings are not necessarily drawn to scale.

FIG. 1 shows a perspective view of a tubular organoid aspect.

FIG. 2 shows a cross-sectional view of the tubular organoid of FIG. 1.

FIG. 3 shows an example apparatus for making tubular structures and tubular organoids.

FIG. 4 shows a side view of example inner and outer nozzles used with an apparatus for making tubular structures and tubular organoids.

FIG. 5 shows an exploded view of example inner and outer nozzles used with an apparatus for making tubular structures and tubular organoids.

FIG. 6 shows a perspective view of inner and outer nozzles coaxially connected.

FIG. 7 shows a perspective cross section of an inner nozzle.

FIG. 8 shows a perspective cross section of an outer nozzle that is positionable around inner nozzle of FIG. 7.

FIG. 9 shows a cross section of an inner nozzle nested within an outer nozzle.

FIG. 10A shows a side view of an example apparatus for making tubular organoids and tubular structures.

FIG. 10B shows a side view of a bottom portion of an example apparatus.

FIG. 10C shows a top down view of the example apparatus from line a-a of FIG. 10A.

FIG. 11 shows a side view of a container.

FIG. 12 shows a side view of an example apparatus for making tubular organoids and tubular structures.

FIG. 13 shows an aspect of a tissue chip.

FIG. 14 illustrates a cannulated tubular organoid or tissue explant in a tissue chip.

FIG. 15 illustrates an assembled tissue chip with a cover compression-fit to the base of the tissue chip. In this aspect the fluid column height is used to control hydrostatic pressure differentials between luminal and extra-luminal compartments.

FIG. 16 shows stills from a video where an extruded tubular structure was manually cannulated with a 34 gauge needle and dye injected into the tube.

FIG. 17 shows tubular organoids of 250 microns in width formed using coaxial extrusion and UV polymerization. The lumens are seeded with Sertoli cells while the outer layer is comprised of cross-linked, gas permeable Gelma.

FIG. 18 shows three tubular organoids in the form of engineered seminiferous tubules, 7-14 days after extrusion. Seeded Sertoli cells proliferated and occupied the luminal space. Tubules with cells self-arranged into a testis-like bundled structure after a week in culture without losing cellular mass. Scale bar=500 microns.

### DETAILED DESCRIPTION

The following description of certain examples of the inventive concepts should not be used to limit the scope of the claims. Other examples, features, aspects, configurations, aspects, and advantages will become apparent to those skilled in the art from the following description. As will be realized, the device and/or methods are capable of other different and obvious aspects, all without departing from the spirit of the inventive concepts. Accordingly, the drawings and descriptions should be regarded as illustrative in nature and not restrictive.

For purposes of this description, certain advantages and novel features of the aspects and configurations of this disclosure are described herein. The described methods, systems, and apparatus should not be construed as limiting in any way. Instead, the present disclosure is directed toward all novel and nonobvious features and aspects of the various disclosed aspects. alone and in various combinations and sub-combinations with one another. The disclosed methods, systems, and apparatus are not limited to any specific aspect, feature, or combination thereof, nor do the disclosed methods, systems, and apparatus require that any one or more specific advantages be present or problems be solved.

Although the operations of exemplary aspects of the disclosed method may be described in a particular, sequential order for convenient presentation, it should be understood that disclosed aspects can encompass an order of operations other than the particular, sequential order disclosed. For example, operations described sequentially may in some cases be rearranged or performed concurrently. Further, descriptions and disclosures provided in association with one particular aspect or implementation are not limited to that aspect or implementation, and may be applied to any aspect or implementation disclosed. It will understood that various changes and additional variations may be made and equivalents may be substituted for elements thereof without departing from the scope of the invention or the inventive concept thereof. Certain aspects and features of any given aspect may be translated to other aspects described herein. In addition, many modifications may be made to adapt a particular situation or device to the teachings of the invention without departing from the essential scope thereof. Therefore, it is intended that the invention not be limited to the particular implementations disclosed herein, but that the invention will include all implementations falling within the scope of the appended claims.

Features, integers, characteristics, compounds, chemical moieties, or groups described in conjunction with a particular aspect, configuration, aspect or example of the invention are to be understood to be applicable to any other aspect, configuration, aspect, or example described herein unless incompatible therewith. All of the features disclosed in this specification (including any accompanying claims, abstract, and drawings), and/or all of the steps of any method or process so disclosed, may be combined in any combination, except combinations where at least some of such features and/or steps are mutually exclusive. The invention is not restricted to the details of any foregoing aspects. The invention extends to any novel one, or any novel combination, of the features disclosed in this specification (including any accompanying claims, abstract, and drawings), or to any novel one, or any novel combination, of the steps of any method or process so disclosed.

Throughout this application, various publications and patent applications may be referenced. The disclosures of these publications in their entireties are hereby incorporated by reference into this application in order to more fully describe the state of the art to which this disclosure pertains. However, it should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions. statements, or other disclosure material set forth herein will only be incorporated to the extent that no conflict arises between that incorporated material and the existing disclosure material.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. The terms “about” and “approximately” are defined as being “close to” as understood by one of ordinary skill in the art. In one non-limiting aspect the terms are defined to be within 10%. In another non-limiting aspect, the terms are defined to be within 5%. In still another non-limiting aspect, the terms are defined to be within 1%.

“Optional” or “optionally” means that the subsequently described event or circumstance may or may not occur, and that the description includes instances where said event or circumstance occurs and instances where it does not.

The terms “coupled,” “connected,” and the like as used herein mean the joining of two members directly or indirectly to one another. Such joining may be stationary (e.g., permanent) or moveable (e.g., removable or releasable). Such joining may be achieved with the two members or the two members and any additional intermediate members being integrally formed as a single unitary body with one another or with the two members or the two members and any additional intermediate members being attached to one another.

Certain terminology is used in the following description for convenience only and is not limiting. The words “right,” “left,” “lower,” and “upper” designate direction in the drawings to which reference is made. The words “inner” and “outer” refer to directions toward and away from, respectively, the geometric center of the described feature or device. The terminology includes the above-listed words, derivatives thereof, and words of similar import.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal aspect. “Such as” is not used in a restrictive sense, but for explanatory purposes.

As used herein, the term “tubular organoid” indicates that the tubular structure contains a cell population. A “tubular structure” may or may not include a cell population.

The disclosed technology addresses a gap in the field of biomedical research. It can be difficult to fabricate small-diameter tubular constructs using currently available technology. Small-diameter tubular constructs could facilitate the engineering of testicular structures, epididymal structures, lymphatic structures, capillaries, oviduct, renal tubules, and alveolar structures, for example (list not intended to be limiting). Each of these tissues form very small diameter tubes, sometimes with only a single layer of epithelial cells. In tissue-engineering, it is understood that 3D architecture can affect the responses of cells. It is important to take the native 3D architecture into account when generating organoids for research and translational applications. Previously, the production of tubular tissue constructs required various methods to pore holes or mold channels in materials such as plastics, PDMS, or hydrogels. Certain limitations persist using these conventional methods, such as limited flow through the tube (poor luminal patency), poor diffusion of nutrients to cells embedded in the tubular wall, and limits on the possible length of the tubes (too short) 3D printing technologies, for example, are limited in the lower diameter of tubes that can be printed. Alginate hydrogels have been utilized by some groups to make small diameter tubes (Sakai et al., Horseradish peroxidase catalase-mediated cell-laden alginate-based hydrogel tube production in two-phase coaxial flow of aqueous solutions for filament-like tissues fabrication. Biofabrication 2013, 5, 1). However, the crosslinking methods typically utilize high calcium concentrations (or high concentrations of another divalent cation). Divalent cations such as calcium play many roles in cell biology—their presence can induce unintended cell signaling pathways (such as reprogramming) or lead to electrical dysfunction. As such, crosslinking alginate in the presence of cells, especially testicular cells, is not ideal. Furthermore, due to currently available alginate crosslinking methods, the thicknesses of the hydrogel are greater than desirable for small tubular tissues It is challenging to create tubular structures with an outer diameter of less than 150 microns using alginate. Alginate is also relatively brittle and/or stiff and as such not as desirable for small tubular tissues that benefit from a higher elasticity. Finally, alginate does not provide an ideal extracellular matrix for cell attachment.

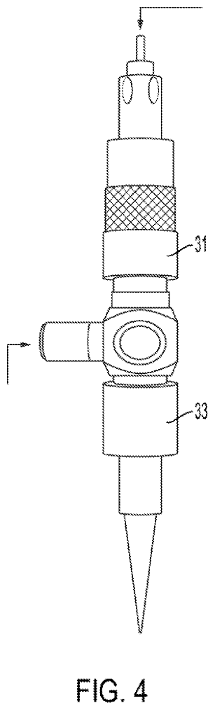

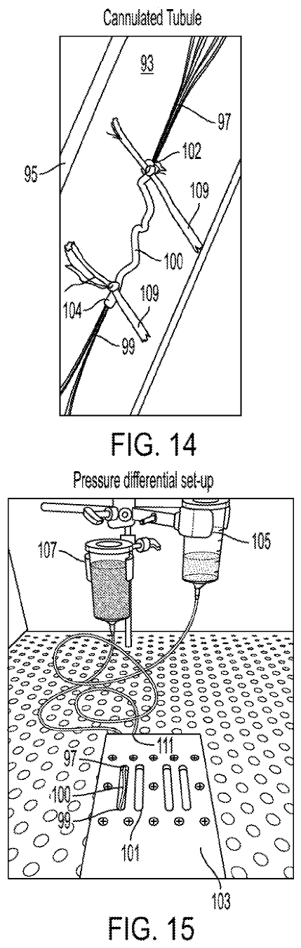

Compared to alginate, gelatin methacryloyl (hereinafter, “gelma”) is more elastic. allowing for cellular remodeling processes (such as the cell-directed bending seen in organoids like that of FIG. 14—this bending could not be achieved using stiffer alginate-based tubes). One research group is printing a mixture of gelatin and gelma to make small tubular structures. Wang et al., Coaxial extrusion of tubular tissue constructs using a gelatin GelMA blend bioink. ACS Biomater. Sci. Eng. 2019, 5, 10, 5514-5524. The protocols utilize thermal cooling of the gelatin to pre-solidify the gelma before photo curing. However, the extruded tubes appear to have a larger standard error of the wall thickness as compared to those disclosed herein (Supplemental FIG. S2 and FIG. 4 of Wang et al, show irregularities in wall structure and the graph in FIG. S2 shows error bars ranging over 200 microns). The tubes described are fairly large (outer diameter of about 600 microns, wall thickness of about 125 microns, and inner diameter of about 400 microns). This is still too large for tissues like capillaries and seminiferous tubules (which have an outer diameter of 180-200 microns and an inner diameter of 40-80 microns). The tubes disclosed herein are much smaller and have fairly consistent radial symmetry. The tubular structures disclosed herein are distinguishable for those reasons, and the methods are distinguishable at least in that they do not require thermal cooling to pre-solidify the wall solution. The wall solution instead directly crosslinks while in laminar flow. The apparatus for making the tubular structures disclosed herein is also different and because of the differences, smaller tubular organoids are made possible. The instant apparatus can make long tubes with consistent inner and outer diameters based on the polymerization properties of the polymer solution alone (like the light curing properties, for example) by optimizing laminar flow pre-and post-extrusion.

The technology described herein utilizes a photocrosslinkable polymer containing extracellular matrix proteins in a laminar flow extrusion and polymerization method using custom designed co-axial extrusion tips. The result is large scale production of organoids with a controlled tubular geometry and controlled, micron-scale inner/outer diameter ratios. To preserve cell viability and function, crosslinking can occur in an environment low or free of divalent cations (such as calcium or magnesium). As such, the organoids are free of divalent cation-based crosslinks. These tubular structures can be seeded with cell types of interest at the time of extrusion in the luminal area and/or tubular wall areas, and/or cells can be seeded in the luminal area and/or extra luminal area after extrusion.

The tubular organoids disclosed herein are useful for chemical toxicity testing and as spermatogenesis models for research or clinical application. Conventional chemical toxicity testing involves complex in vivo studies that are time consuming and costly. With an extensive backlog of chemicals already in use needing toxicity testing to inform regulatory decisions, it is important to develop in vitro models representative of complex cellular interactions and validated for relevant higher throughput toxicological testing. Current in-vitro models for spermatogenesis are limited to testis explants that are not proliferative or consistent in spermatogenic output, and underdeveloped organoids do not recapitulate the complex microenvironment of the seminiferous epithelium nor produce functional sperm. These novel approaches provide improved in-vitro systems more closely resembling in-vivo physiology and allowing development of fertility preservation and animal free toxicant screening models.

Tubule organoids can be cultured in any available fashion, including free floating in media of traditional cell culture methods, or incorporated in more advanced culture systems (such as, but not limited to, tissue-chips). A number of factors facilitate the production of the tubular organoids, including (but not limited to), the extrusion head geometry, extrusion materials, environmental conditions, and curing conditions. With respect to the extrusion head geometry, the diameter of the outer extrusion nozzle tip and total (inner and outer material) extrusion rate affect the extruded tube outer diameter. The inner extrusion nozzle diameter. distance from outer extrusion tip (to allow for laminar flow stability), ratio of inner and outer/nested cross-sectional area at the point of inner extrusion tip end, and flow rates that match ratios closely enough to prevent chaotic flow disruptions all affect the reproducibility of results for a given tube geometry. The velocity of extruded material from nested extrusion tips can be matched for optimum flow.

With respect to extrusion materials, the extrusion system works with high or low viscosity materials. Flow properties can be improved by density matching extruded materials. Matching the viscosity and/or density of the inner (luminal) solution, the outer (wall) solutions, and/or the solution of any of the nested layers of the structure can improves the production of tubular organoids. In one example, such as may be used to construct tubular organoids mimicking the seminiferous epithelium, the wall solution can be gelatin methacryloyl (hereinafter abbreviated gelma) and the luminal solution can be gelatin, each with matched weight volume which in some aspects is 5-15%. Other concentrations and other inner and outer extrusion solutions, such as PVP containing buffer solutions and high-density sugar solutions, can also work. The wall and luminal extrusion solutions can be selected to suit the particular bioengineering application. The wall solutions may contain additional materials to alter extruded tube properties such as porosity, stiffness, and cellular responses to included factors. Addatives may include other photoactivatable material such as PEGDA or non-photoactivatable materials such as PEG, decellularized extracellular matrix or purified factors such as fibronectin, modified signaling peptides that may be chemically linked to another material such as the GELMA, additional hydrogels, or additives. These may be included in the tubular wall materials in one or more layers. Pumps (such as, but not limited to, syringe pumps) can be used in a thermally controlled environment to extrude the wall and luminal solutions through the extrusion tip and into a tall column of extrusion buffer. In some scenarios, the tips of the inner and outer extrusion nozzles are directly submerged within the extrusion buffer. Multiple factors contribute to sufficient curing that preserves the patency of the organoid lumen, including, but not limited to, the light wavelength, the light intensity, distance of initial curing from extrusion tip (i.e., the distance the tubular structure sinks before curing), total time of light exposure, and the distance between the container and the light source.

Current coaxial extrusion systems are larger scale and can be unsuitable for use with gelma in particular. For example, it is difficult to extrude small scale alginate tubes using the methods and apparatuses disclosed herein because alginate is relatively buoyant, and not as amenable to laminar flow. Printing of larger scale gelma tubes has been reported. Ding et al., Printability Study of Bioprinted Tubular Structures Using Liquid Hydrogel Precursors in a Support Bath. Appl. Sci. 2018, 8, 3, 403. These methods relied on partial gelation due to cooler temperature. In methods that are standard for 3D printers, the extruded gelma was partially solidified prior to extrusion in air. Tubes were built by coiling the partially solidified gelma “ropes,” then subsequently crosslinking by photoactivation. However, tubes produced by these methods cannot be made in the size range needed to replicate small diameter tubes of tissues mentioned above. The methods disclosed herein describe extrusion into a liquid bath, facilitating the laminar flow and photocuring that leads to uniform tube sizing. The methods facilitate curing of very small diameter shapes, limited only by the ability to supply small diameter extrusion tips. Current manufacturing technology allows for extrusion tip pore sizes of as small as 40-50 micron diameter, and will likely get smaller as manufacturing technology progresses. In practice, the lower limit of tubes produced for use in applications where cells will be co-extruded may be limited by sheer-stress limits for cell survivability. As discussed in the examples, the methods disclosed herein have allowed for culture of extruded constructs of tubes within size ranges typically seen in the seminiferous epithelium across species, thus demonstrating good cell survivability of this system.

The apparatus described herein to fabricate the tubular organoids can find commercial utility in multiple industries. Labs that work on bioengineering any tubular tissue types could benefit from such an apparatus because it would provide consistency that is difficult to achieve with custom-built systems that use off-the-shelf components. The apparatus might also be useful in toxicant screening by national toxicology programs and toxicity testing labs. The apparatus might further be useful in clinical settings, such as (but not limited to) assisted reproductive technologies clinics.

The following patents and patent application publications may be pertinent to the disclosure and are incorporated by reference herein: U.S. Pat. Nos. 10,208,289; 10,099,417: 9,926,534; 9,157,060; 9,573,311: 9,067,204: 8,398,935.

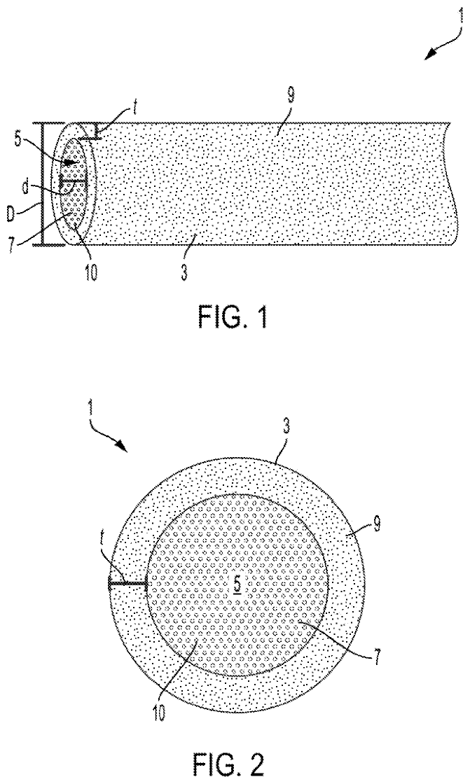

A tubular organoid 1 is depicted in FIG. 1. The tubular organoid 1 includes a tubular wall 3, a lumen 5 defined by the tubular wall 3, and a luminal material 7 within the lumen 5. Note that the tubular wall can be further composed of one or more nested radial layers. FIG. 2 shows a cross section of the tubular organoid of FIG. 1 with a single wall layer. In the example of FIGS. 1-2, a first cell population 9 lives in the tubular wall 3 and a second cell population 10 is suspended or encapsulated within the luminal material 7. Alternatively or in addition, cells can be seeded onto the exterior surfaces of the tubes (smooth muscle cells, as one example).

In some aspects, the tubular organoid can be tightly bundled and/or coiled. See, for example, the coiled tubular organoid depicted in FIG. 14, which depicts the state of a tubular organoid after 7-14 days of cell culture under conditions that promote coiling of the tubular organoid. With a coiled tubular organoid, the tubular structure comprises a plurality of bends, resulting in a high tortuosity when compared to a cylindrical tube. Here, the cell population 9 of seminiferous epithelial cells remodeled the originally cylindrical shape of the tubular organoid 1 to the bundled tubular organoid 101 that is shown in FIG. 14.

The tubular organoid 1 can include one or more cell populations housed in one or more nested radial layers of the tubular wall 3, in the luminal material 7, or in both the tubular wall 3 and the luminal material 7 (and/or on the surface of the tubular inner or outer walls). The devices and methods disclosed herein can be used to engineer any small tubular organ, including, but not limited to, vasculature, lymphatic vessels, male and female reproductive ducts (for example, seminiferous tubules, fallopian tubes, efferent ducts, epididymal tubules, vans deferens, etc.), biliary and pancreatic ducts, ureters, renal tubules, and urethras. As such, the cell population can include any number of cell types, including, but not limited to, endothelial cells, epithelial cells, smooth muscle cells, and any cell type naturally present in any of the aforementioned small tubular organs. The disclosure is intended to cover adult, progenitor, and stem cell types (of any developmental stage) of the aforementioned small tubular organs. The cell population can be autologous, allogenic, or xenogenic.

In some aspects, the tubular organoid produces germ cells. In some aspects, the tubular organoid produces haploid germ cells. In some aspects, the cell population comprises testicular cells. In some aspects, the tubular organoid mimics a testicle, or a seminiferous tubule, in that it produces sperm cells. In some aspects, the tubular organoid produces haploid male germ cells.

A length of the tubular organoid 1 can be measured between a first end of the tubular wall 3 to a second end of the tubular wall 3, and can range from less than a centimeter to 10 meters or more.

The outer surfaces of tubular wall 3 define the outer diameter D of the tubular organoid 1. The outer diameter D can range from about 1 micron to about 1000 microns (including, for example, about 1 micron, about 10 microns, about 20 microns, about 30 microns, about 40 ) microns, about 50 microns, about 60 microns, about 70 microns, about 80 microns, about 90 ) microns, about 100 microns, about 110 microns, about 120 microns, about 130 microns, about 140 microns, about 150 microns, about 160 microns, about 170 microns, about 180 microns, about 190 microns, about 200 microns, about 210 microns, about 220 microns, about 230 microns, about 240 microns, about 250 microns, about 260 microns, about 270 microns, about 280 microns, about 290 microns, about 300 microns, about 350 microns, about 400 microns, about 450 microns, about 500 microns, about 550 microns, about 600 microns, about 650 microns, about 700 microns, about 750 microns, about 800 microns, about 850 microns, about 900 microns, about 950 microns, and about 1000 microns). In some aspects, the outer diameter D can be from about 50 microns to about 300 microns.

The outer diameter D is fairly consistent across the length of the tubular organoid 1. The standard error of the outer diameter D can be, for example, less than 10% of the outer diameter D, including less than about 10%, less than about 9%, less than about 8%, less than about 7%, less than about 6%. less than about 5%, less than about 4%, less than about 3%, less than about 2%, and less than about 1%. In some examples, the standard error of the outer diameter D is from about 3% to about 10% of the outer diameter D.

The total radial wall thickness t is approximately one half the difference between the outer diameter D and the inner, luminal diameter d. The tubular wall 3 ranges in thickness t from about 1 micron to about 500 microns (including, for example, about 1 micron, about 5 microns, about 10 microns, about 15 microns, about 20 microns, about 25 microns, about 30 microns, about 35 microns, about 40 microns, about 45 microns, about 50 microns, about 55 microns, about 60) microns, about 65 microns, about 70 microns, about 75 microns, about 80 microns, about 85 microns, about 90 microns, about 95 microns, about 100 microns, about 120 microns, about 130 microns, about 140 microns, about 150 microns, about 160 microns, about 170 microns, about 180 microns, about 190 microns, about 200 microns, about 210 microns, about 220 microns, about 230 microns, about 240 microns, about 250 microns, about 260 microns, about 270 microns, about 280 microns, about 290 microns, about 300 microns, about 310 microns, about 320 microns, about 330 microns, about 340 microns, about 350 microns, about 360 microns, about 370 microns, about 380 microns, about 390 microns, about 400 microns, about 410 microns, about 420 microns, about 430 microns, about 440 microns, about 450 microns, about 460 microns, about 470 microns, about 480 microns, about 490 microns, and about 500 microns). In some aspects, the tubular wall 3 ranges in thickness t from about 5 microns to about 200 microns.

In some aspects, the tubular wall may be comprised of one or more nested radial layers generated by nested extrusion tips or nozzles. The thickness of an individual wall layer can be about 0.1 microns to about 200 microns.

The thickness of the wall t is fairly consistent across the length of the tubular organoid 1. That is, the standard of error of thicknesses data points taken at various distances from a first end of the tubular organoid 1 is advantageously low as compared to the standard error of tubular structures formed by more conventional bioprinting methods. For example, in some aspects, the standard error of the radial thickness can be from about 3 percent to about 20 percent (including about 3 percent, about 4 percent, about 5 percent, about 6 percent, about 7 percent, about 8percent, about 9 percent, about 10 percent, about 11 percent, about 12 percent, about 13 percent, about 14 percent, about 15 percent, about 16 percent, about 17 percent, about 18 percent, about 19 percent, and about 20 percent). In some aspects, an approximately 200 micron outer diameter tube with approximately 50 micron thick walls can have a standard error of the thickness t that is less than 20 microns, less than 12 microns, or less than 8 microns. In one non-limiting example. tubular organoids averaging 204 microns in luminal diameter and 56 microns in wall thickness had a standard deviation of 12 microns.

The luminal diameter d of the tubular organoid (also known as the inner diameter of the tubular organoid) is defined by the inner surfaces of the tubular wall 3and is measured across the lumen 5, as shown in FIG. 1. The luminal diameter d can range from 1 micron to 500 microns (including, for example, about 1 micron, about 10 microns, about 20 microns, about 30 microns, about 40 microns, about 50 microns, about 60 microns, about 70 microns, about 80 microns, about 90 microns, about 100 microns, about 150 microns, about 200 microns, about 250 microns, about 300 microns, about 350 microns, about 400 microns, about 450 microns, and about 500 microns). In some aspects, the luminal diameter d can range from about 40 microns to about 200 microns.

The luminal diameter d is fairly consistent across the length of the tubular organoid 1. That is, the standard of error of luminal diameter data points taken at various distances from a first end of the tubular organoid 1 is relatively low as compared to the standard error of luminal diameters of tubular structures formed by more conventional bioprinting methods. In some aspects, the standard error of the luminal diameter d is from about 1% to about 15% (including about 1%, about 2%, about 3%, about 4%, about 5%, about 6%, about 7%, about 8%, about 9%, about 10%, about 11%, about 12%, about 13%, about 14%, and about 15%). In some examples, the standard error of the luminal diameter d can be less than 10%, less than 6%, or less than 4%.

The tubular wall 3 can be formed of any number of hydrogel wall materials. The hydrogel can include natural polymers or synthetic (non-natural) polymers. The hydrogel can be a non-biodegradable hydrogel, a natural biodegradable hydrogel, and/or a synthetic biodegradable hydrogel. In some aspects, the hydrogel wall material can be free of divalent cation-based crosslinks. As discussed above, a hydrogel without divalent cation-based crosslinks gives better cell outcomes in some applications than, for example, conventional alginate hydrogels that are ionically crosslinked using calcium, barium, strontium, and/or magnesium cations which can cause electrical dysfunction and/or induce unintended cell signaling pathways. Likewise, the hydrogel wall material can be free of alginate in some aspects. In some aspects, the hydrogel material is gelatin methacryloyl (gelma), which has a polymer structure that is free of divalent cation-based crosslinks.

Altering molecular weights, block structures, degradable linkages, and cross-linking modes can influence strength, elasticity, and degradation properties of the hydrogels. Hydrogels can also be modified with functional groups for covalently attaching a variety of proteins (e.g., collagen) or compounds such as therapeutic agents. Molecules which can be incorporated into the hydrogel include, but are not limited to, PEGDA, PEG, glycoproteins, fibronectin; peptides and proteins; carbohydrates (both simple and/or complex); proteoglycans; antigens; oligonucleotides (sense and/or antisense DNA and/or RNA); antibodies and growth factors. In one aspect, the hydrogel includes molecules that aid in the growth and proliferation of a cell when cultured in or on the hydrogel. Non-limiting examples of such molecules can include proteins, peptides, supplements, small molecule inhibitors, glycosaminoglycans, growth factors, nucleic acid sequences, and combinations thereof. These molecules can be a growth factor. Factors and components added to the extruded materials may be added to one or more of the radial layers of the wall or lumen, and may differ between layers as suited for the application.

In some aspects, the hydrogel wall material can be a gelatin hydrogel, a collagen hydrogel, a fibrin hydrogel, a polysaccharide hydrogel, an alginate hydrogel, a laminin hydrogel, a fibronectin hydrogel, a laminin hydrogel, a vitronectin hydrogel, a polyethylene glycol hydrogel, a gelatin methacryloyl hydrogel, or a combination thereof. In some aspects, the hydrogel includes decellularized extracellular matrix. For example, the hydrogel can include tissue-specific decellularized extracellular matrix in order to provide specific chemical and mechanical cues to particular cell types. In some aspects, the hydrogel is manufactured from biodegradable materials which degrade in vivo or in vitro, at a sufficiently slow rate to allow the cells to proliferate. Commercially available hydrogels include, but are not limited to, MATRIGEL® and NOVOGEL2®. However, the disclosure is not intended to be limited to any particular hydrogel wall material.

In certain aspects, the hydrogel includes a self-assembly peptide, a fibrin, an alginate, an agarose, a hyaluronan, a hyaluronic acid, a chitosan, a chondroitin sulfate, a polyethylene oxide (PEO), a poly(ethylene glycol) (PEG), a collagen type L a collagen type II hydrogel, or combination thereof. In a further aspect, the hydrogel composition includes a hydrogel selected from the following: self-assembly peptide, fibrin, alginate, agarose, hyaluronan, hyaluronic acid, chitosan, chondroitin sulfate, collagen type L collagen type II, and combinations thereof. In additional aspects, the hydrogel includes bioabsorbable materials selected from gelatin, alginic acid, chitin, chitosan, dextran, polyamino acids, polylysine, and copolymers of these materials.

A hydrogel can be prepared by crosslinking hydrophilic biopolymers or synthetic polymers. Examples of the hydrogels formed from physical or chemical crosslinking of hydrophilic biopolymers include, but are not limited to, gelatin, hyaluronans, chitosans, alginates, collagen, dextran, pectin, carrageenan, polylysine, and/or agarose. Examples of hydrogels based on chemical or physical crosslinking of synthetic polymers include but are not limited to (meth)acrylate-oligolactide-PEO-oligolactide-(meth)acrylate, poly(ethylene glycol) (PEO), poly(propylene glycol) (PPO), PEO-PPO-PEO copolymers (Pluronics), poly(phosphazene), poly(methacrylates), poly(N-vinylpyrrolidone), PL(G) A-PEO-PL(G)A copolymers, polyethylene imine), etc. Additional photoactivatable materials such as (but not limited to) PEGDA may be added. These hydrogels can be modified with fibronectin, laminin, or vitronectin, for example.

The wall solution can also include one or more photoinitiators. Illustrative, but non-limiting, examples of photoinitiators include lithium phenyl-2,4,6-trimethylbenzoylphosphinate (LAP) photoinitiator, 2-hydroxy-2-methyl propiophenone (e.g., Irgacure™ 1173, Darocur™ 1173), and combinations thereof. A concentration of the one or more photoinitiators in the hydrogel forming solution can be from about 0.0001 wt % to about 1 wt %, such as from about 0.001 wt % to about 0.9 wt %, such as from about 0.01 wt % to about 0.5 wt %, such as from about 0.05 wt % to about 0.1 wt %, based on the total wt % of the hydrogel forming solution. Higher or lower concentrations of the one or more photoinitiators can be used depending on, e.g., the application or desired results.

Polymerizing initiators include electromechanical radiation. Initiation of polymerization may be accomplished by irradiation with ultraviolet or visible light, such as between about 350 nanometers to about 740 nanometers, such as between about 350 nanometers to about 700 nanometers, such as between about 365 nanometersto about 514 nm, such as between about 380 nm to about 405 nm. In some aspects, polymerization can also include cross-linking with UVA, UVB, and/or UVC light.

The mechanical properties of a cross-linked polymer matrix, such as a hydrogel, may also be related to pore structure. Hydrogels with different mechanical properties may be desirable depending on the desired clinical application.

The tubular organoid also includes a luminal material 7 within the lumen 5 of the tubular organoid 1. The luminal material serves to prevent the tubular walls 3 from collapsing during coextrusion of the tubular organoid 1. The luminal material can be any solution, especially any solution that is cell compatible. In some aspects, the luminal material 7 is a solution of a similar density and viscosity as the wall solution (prior to its polymerization into the tubular wall). The luminal material 7 can be, for example, sucrose, gelatin, saline, PVP or PVA containing buffers, or any compatiblebuffer where the density is sufficient that extruded tubes do not disturb laminar flow; float in the extrusion bath where polymerization occurs outside the outer tip, or otherwise interfere with laminar flow before curing. In some aspects, the luminal material 7 is aqueous and diffuses out over time, leaving cells behind within the lumen. In other aspects, the luminal material 7 is polymerized within the tubular walls 3. The luminal material 7 may be, for example, a biodegradable polymer that degrades at a faster rate than the tubular walls 3 (which may or may not be biodegradable)

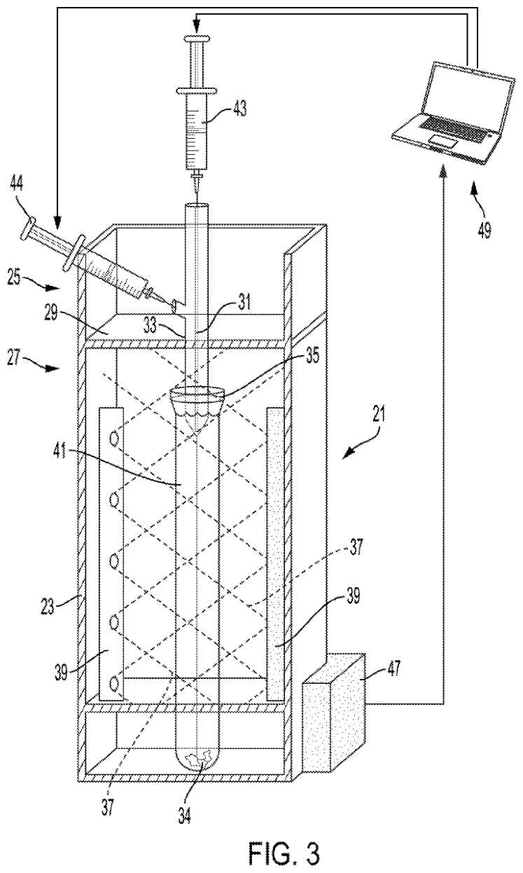

The extrusion buffer can be any aqueous media, especially media that is used in cell culture applications. This could be simple buffer solutions such as PBS, HBSS, F-12 medium, MEMalpha medium, for example. Calcium and magnesium free extrusion buffer can be advantageous in some aspects. When tubes are extruded without cells, the extrusion buffer can be water or any medium compatible with the wall materials. Apparatuses for coextruding tubular structures are disclosed herein. An example of such an apparatus 21 is shown in FIG. 3. The apparatus includes a housing 23, an upper compartment 25, a lower compartment 27, and a frame 29 dividing photo-sensitive materials in the upper compartment 25 from the light source in the lower compartment 27. The apparatus 21 further includes an outer extrusion nozzle 33 removably positioned within the housing 23 and extending from the upper compartment 25 to the lower compartment 27 such that only the extrusion tip my be exposed to activating light sources. The apparatus 21 further includes one or more nested inner extrusion nozzles 31 removably positioned coaxially within the outer extrusion nozzle 33.

In some aspects, the inner and/or outer extrusion nozzles are removable. Single piece nested coaxial nozzles can be manufactured through additive processes. Due to their removability, inner and outer nozzles 31, 33 are advantageously able to be removed for cleaning and/or to be replaced by nozzles of a different interior pore size for various applications. That is, the sizes of the nozzles can be varied to create tubular structures 34 of different sizes for different types of organoids. However, removability is not a required feature of the nozzles and the disclosure is not intended to be so limited. In some aspects, the inner or outer nozzle may be permanently coupled to the apparatus.

Pumps are attached to the inner and outer nozzles 31, 33 to introduce luminal and wall solutions, respectively. For example, in FIG. 3, a pump 44 is fluidically connected to an interior pore of the outer extrusion nozzle 33, and a second pump 43 is fluidically connected to an interior pore of the inner extrusion nozzle 31. In some aspects, the pumps 43, 44, can be connected to a control system (in a wired or wireless fashion), such as a computing device, user interface, or processor 49. The processor 49 permits automated control of the pumps and the fluid flow. Alternatively, the pumps 43, 44 can be manually controlled, for example, using a power switch directly on the pump. The pumps 43, 44 can be any dynamic or displacement pump, for example, a syringe pump, a peristaltic pump, a positive displacement pump, a hydraulic pump, and/or a pneumatic pump. In some aspects of the invention, just one pump can be used to control flow into both nozzles 31, 33. For example with syringe pumps, using differing diameter syringes can be used to match or alter flow rate from a single pump.

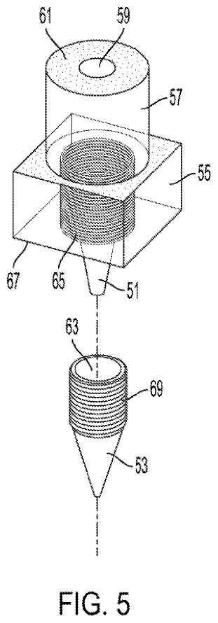

FIG. 3 shows the cross-sectional view of an outer nozzle 33 supported by the frame 29, with inner nozzle 31 coaxially inserted into the outer nozzle 33. FIG. 4 shows an exterior view of a similar configuration, where inner nozzle 31 is coaxially inserted into outer nozzle 33 (arrows show the direction of fluid flow from the respective pump into the nozzle). FIG. 5 shows a slightly different configuration of coaxial inner and outer nozzles 51, 53. In FIG. 5, the inner nozzle 51 is constructed, removably or irremovably, as part of a block 55. Block 55 has a cavity 65 extending upward from its lower surface 67. The walls surrounding cavity 65 have one or more connecting features, such as (but not limited to) threads, grooves, indentations, or compression features. The connecting features on the walls surrounding cavity 65 mate with one or more connecting features on the outer surface 69 of outer nozzle 53. As such, outer nozzle 53 can be removably attached to block 55 in such a way that it is coaxial with inner nozzle 51. Each of these specific features can be modified for need and additional tips or nozzles can be nested to generate additional tubule layers. Block 55 can advantageously be rested upon frame 29, for example, within ridges or guides on the upper surface of frame 29 to center the block 55 and thus, the nozzles 51, 53 above the container 35.

A pipe 57 protrudes from the top of block 55—the interior pore 59 of the pipe 57 is (or is fluidically connected to) the interior pore of the interior nozzle 51. The pipe 57 can be smooth, contain barbs, threads, slip fit/compression fit mechanisms, or any other means of connecting fluidically to tubing and/or pumps. The wall of pipe 57 includes a channel 61 that fluidically connects to the interior pore 63 of the outer nozzle 53. Channel 61 can also be smooth, contain barbs, threads, slip fit/compression fit mechanisms, or any other means of connecting fluidically to tubing and/or pumps. Pumps for the outer wall layer and luminal solutions (such as 43 and 44 shown in FIG. 3) can be fluidically connected to channel 61 and pore 59 for delivery to the outer nozzle 53 and inner nozzle 51, respectively.

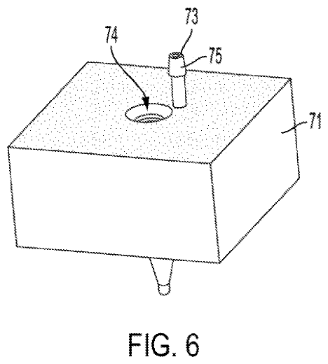

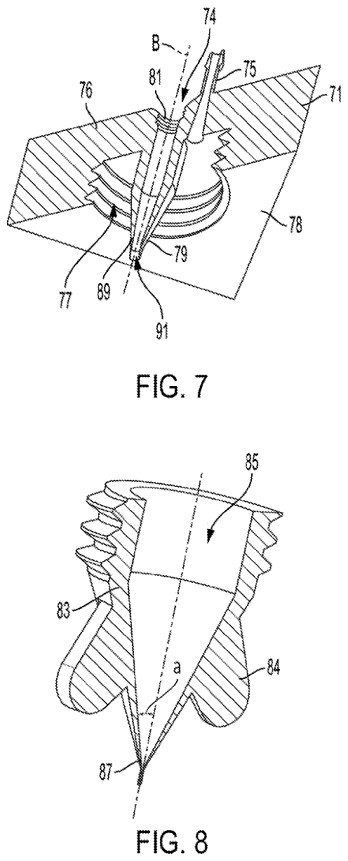

FIG. 6 shows another aspect of a block with variations on ways to attach tubing and/or pumps. Channel 73 extends upward from block 71 to create a port 75 for affixing tubing to a pump. Like channel 61 in FIG. 5, channel 73 of FIG. 6 delivers extrusion materials to an outer nozzle. Like pore 59 in FIG. 5, upper opening 74 of FIG. 6 delivers extrusion materials to an inner nozzle. A cross section of this aspect is shown in FIG. 7. Like the aspect of FIG. 5, the block 71 includes a cavity 77 extending upward from a lower surface 78 of the block 71. A upper opening 74 extends downward from the upper surface 76 of block 71 to become the interior pore of inner nozzle 79. Connecting features, such as threads 81, are present at the upper opening 74 of the interior pore of inner nozzle 79 for affixing tubing to the pump. Any and all connections for tubing and/or pipes can be smooth, contain barbs, threads, slip fit/compression fit mechanisms, or any other means of fluidic connection.

The outer nozzle 83 that connects to block 71 is shown in FIG. 8. This aspect contains grips 84 to facilitate changing of nozzles. Other mechanisms to facilitate changing of nozzles are a wrench nut shape, inclusion of “key” holes or spanner wrench holes.

The interior pore of outer nozzle tapers at an angle α, progressively narrowing the diameter of the interior pore 85 of the outer nozzle 83. At the tip 87 of outer nozzle 83, the diameter of interior pore 85 is less than about 1000 microns (including, for example, less than 1000 microns, less than 900 microns, less than 800 microns, less than 700 microns, less than 600microns, less than 500 microns). In some aspects, the diameter of interior pore 85 of the outer nozzle is less than about 400 microns (including, for example, less than 400, less than 380, less than 360, less than 340), less than 320, less than 300, less than 280, less than 260, less than 240), less than 220, less than 200, less than 180, less than 160, less than 140, less than 120, less than 100, less than 80, less than 60, less than 40, and less than about 20 microns).

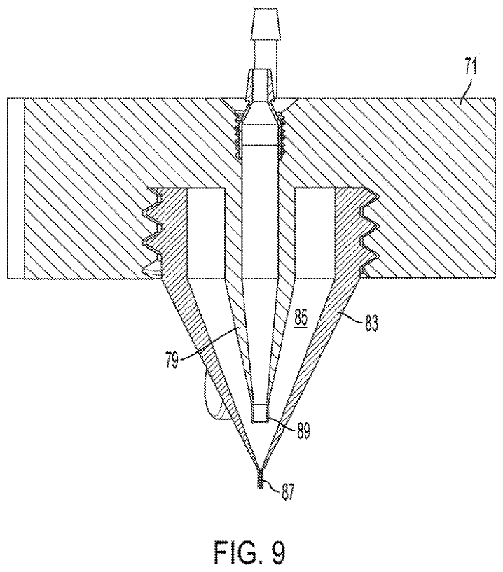

FIG. 9 shows a cross section of the outer nozzle 83 coupled to the block 71 such that inner nozzle 79 is nested inside outer nozzle 83. When the inner nozzle 79 and the outer nozzle 83 are coaxially coupled, the inner nozzle tip 89 ends before the outer nozzle tip 87 at the end of interior pore 85, allowing for inner nozzle tip 89 to be a larger diameter than outer nozzle tip 87. As such, luminal solution exiting the inner nozzle tip 89 is carried with the wall solution flowing through the interior pore 85 of the outer nozzle 83 and out the outer nozzle tip 87, into the buffer solution in the container 35, as described below. Having a larger inner nozzle tip 89 than outer nozzle tip 87 lessons the sheer forces on cells in the luminal compartment and reduces clogging of the inner nozzle 79. Sheer is also reduced by the “carrying” effect from the wall solution flowing around the inner nozzle tip 89. In aspects with additional nested tips/nozzles, the outer tip solution can be non-polymerizing to aid in reduction of sheer on inner layers.

The nozzles can be designed to create desired wall; lumen diameter ratios in the tubular structures 34. For example, in addition to relative flow rates of the two solutions, the ratio of the area of outer nozzle pore 85 to the area of inner nozzle pore 91, measured at the plane of inner tip extrusion, can be chosen to match the desired wall; lumen diameter ratio of the tubular structure 34.

Tapering of the outer nozzle 83 facilitates extrusion of tubular structures and reduction of size. The inner nozzle 79 can be tapered as shown, or it can be straight. The continued tapering of the outer tip compresses material to final size. The outer tip end diameter plays a large role in setting final tubular structure size. In aspects where the outer tip extrudes non-polymerizing material, the volume of the outer material extruded relative to tubular wall and lumen materials will allow size reduction of the final tubular product.

The nozzles can be fabricated by a number of manufacturing techniques, for example micro machining techniques that may include for example milling, and/or drilling. The nozzles may also be made by molding. The nozzles may also be made by additive manufacturing such as 3D laser sintering, or by 3D lithography. In some aspects, the nozzles are formed of moldable or printable materials, such as, but not limited to, stainless steel, acrylic, nylon, and/or 3D printable materials.

In some aspects, such as shown in FIG. 3, the outer extrusion nozzle 33 can be supported by the frame 29 that divides the upper compartment 25 from the lower compartment 27. The inner and outer extrusion nozzles 31, 33 are positioned above a column shaped container 35. The column-shaped container 35 has light permeable walls such that light rays 37 can be transmitted from a light source 39 (also positioned in the housing 23) and into the column-shaped container 35. The housing 23 can include access doors in the upper and lower compartments for exchanging the nozzles and/or the container 35.

The outer extrusion nozzle 33 can be positioned, in some aspects, with the tip of the nozzle submerged in the buffer 41 of the container 35. Tip submersion may be advantageous in some applications (for example, for very small tubes) to mitigate obstacles that might otherwise be faced in breaking the surface tension of the extrusion buffer. However, tip submersion may not be necessary in all applications. Larger tubes and/or faster extrusion rates can also mitigate the issue of breaking the surface tension. Photo-curing can also occur in the end of the outer extrusion tip in applications where the outer material is non-photoactivating.

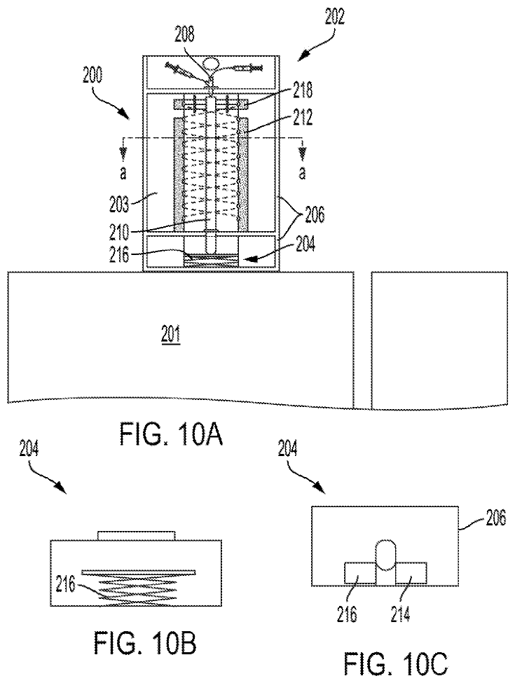

The light source 39 is oriented such that the light rays 37 polymerize the wall solution as it is extruded from outer nozzle 33 and is sinking through the buffer solution 41. The intensity, distance, and angle of the light source 39 is set to predetermined values to optimize the timing of polymerization of the tubular structure 34. In some aspects, the lights can be positioned on a swivel, and with mechanisms to allow adjustable spread angles. See, for example, FIG. 10, showing light rays 37 coming from light sources 39. The light source position and angles can be varied hit the buffer-container 35 at different places along its length, thus changing the timing of polymerization of tubular structures sinking through the column. While the light source 39 of FIG. 3 is depicted as inside the walls of lower compartment 27, it is also envisioned that the light source 39 can be positioned outside the walls of housing 23 if the walls are formed of a light permeable material. The walls of the upper compartment 25 can be opaque to prevent light rays 37 from travelling through the frame 29 and polymerizing the wall solution before it is extruded. In some aspects there may be a single point source of light for photo-polymerization.

In some aspects, the light source 39 can be an elongated array with a series of lights positioned alongside the column-shaped container as shown in FIG. 3. The lights can be LED lights in some aspects. The wavelength can be, for example, UVA, violet, or blue light. The wavelength can be, for example, between about 350 nanometers to about 740 nanometers, between about 350 nanometers to about 700 nanometers, between about 365 nanometers to about 514 nm, and/or between about 380 nm to about 405 nm.

The elongated nature of the container 35 allows for light to hit the sinking wall solution for a sufficient duration to polymerize the wall solution for some applications. In this manner, the walls of tubular structures 34 do not collapse when they hit the bottom of the container 35. The tubular shape is achieved, in part, by the submersion of nozzles 31, 33 into buffer 41 which ensures that the wall solution flows easily into the buffer 41. The polymerizing wall solution retains its tubular shape as it becomes tubular structure 34 because it does not hit the surface of buffer 41 before it polymerizes. The width of the container 35 is also important it is narrow enough to reduce turbulence that might distort the shape of the wall solution as it sinks and polymerizes. In some aspects, the container has a length between about 5 centimeters to about 100 centimeters (including, for example, about 5 centimeters, about 10 centimeters, about 15 centimeters, about 20 centimeters, about 25 centimeters, about 30 centimeters, about 35 centimeters, about 40 centimeters, about 45 centimeters, about 50 centimeters, about 55 centimeters, about 60 centimeters, about 65 centimeters, about 70 centimeters, about 75 centimeters, about 80 centimeters, about 85 centimeters, about 90 centimeters, about 95 centimeters, and about 100 centimeters). In some aspects, the container 35 has a diameter of about 0.5 centimeters to about 10 centimeters (including about 0.5 centimeters, about 1 centimeter, about 2 centimeters, about 3 centimeters, about 4 centimeters, about 5 centimeters, about 6 centimeters, about 7 centimeters, about 8 centimeters, about 9 centimeters, and about 10 centimeters). In some aspects, the container 35 is made of quartz, UV transmissible acrylic, or other UV transparent materials. In other aspects the outer tip or nozzle is made of such UV-transmissible materials to allow photo-curing.

Environmental conditions within the housing 23 (gas levels, temperature, humidity, for example) can be regulated for heightened cell viability and polymerization rates via an environmental regulation system 47. The environmental regulation system 47 can be connected to a control system (in a wired or wireless fashion), such as a computing device, user interface, or processor 49. The processor 49 permits automated control of the environmental regulation system. The environmental regulation system 47 can include, for example, a light ballast, a light intensity regulator, a gas regulator, a heating component, a cooling component, a water reservoir, a control panel, and/or a user interface for selecting the desired conditions. Components of the environmental regulation system 47 can be positioned at any position around the housing 23.

Additional variations and aspects of the apparatus for making tubular structures are described in the Example section and shown in FIGS. 10-12.

Tissue chips and bioreactors can be used to culture tubular structures or tubular organoids (tubular structures containing cells). A tissue chip or bioreactor facilitates cannulation of the tubular structures in a manner that enables separation of the luminal and extra-luminal compartments (the luminal compartment being the lumen of the tubular structure), prevents mixing of fluids between the compartments, and facilitates the maintenance of distinct conditions between the compartments. The luminal compartment can be fluidically coupled to one flow system whereas the extra-luminal compartment can be connected to a different flow system than the luminal compartment (or the extra-luminal compartment can be an open bath). The high patency of the tubular structure wall prevents mixing between the luminal and extra-luminal compartments. In this arrangement, hydrostatic pressure, media composition, fluid flow rates, sheer stresses, and media additives such as growth factors or test compounds (for toxicity or male fertility, as some examples) can be different in the luminal compartment and in the extra-luminal compartment. Media from each compartment can be sampled to test for differential accumulation, metabolism and diffusion. The tubular structures may be pre-incorporated into chips on cannula, or embedded into a canulated frame. These tissue chips can find commercial utility in multiple industries as cell-free (fit to application) or pre-cell-seeded products. Bioengineering endeavors for any tubular tissue type could benefit from such an apparatus because it would provide consistency that is difficult to achieve with custom-built systems that use off-the-shelf components. The apparatus might also be useful in toxicant screening by national toxicology programs and toxicity testing labs. The apparatus might further be useful in clinical settings, such as (but not limited to) assisted reproductive technologies clinics.

Connecting tubular structures by cannulation into a culture system allows for controlled hydrostatic and hydrodynamic regulation of the luminal and extra-luminal compartments and enables the testing of cellular responses to these forces. This system allows for better mimic of interstitial fluid flow surrounding such tubular tissues such as lymphatics, vasculature, and seminiferous epithelium in a way that is limited in systems where “tubular” pores are bored or cast into hydrogel or other matrix. Differential pressures can be maintained in the inner (luminal) and outer extra-luminal compartments and the role of hydrostatic pressure differentials in tissue responses/health in culture systems can be investigated. As one example, using syringe pumps capable of sub-microliter/min flow, the flow through the tubes can also be regulated to match physiologically relevant flow rates and test (and apply based on results) dynamic flow effects. The bath around the tubular structure (extra-luminal compartment) can be representative of the interstitial region. Interstitial fluid dynamics are key for nutrient/waste transport from vasculature and lymphatics in most tissue types, so a better understanding of interstitial fluid dynamics would advance the fields of interest.

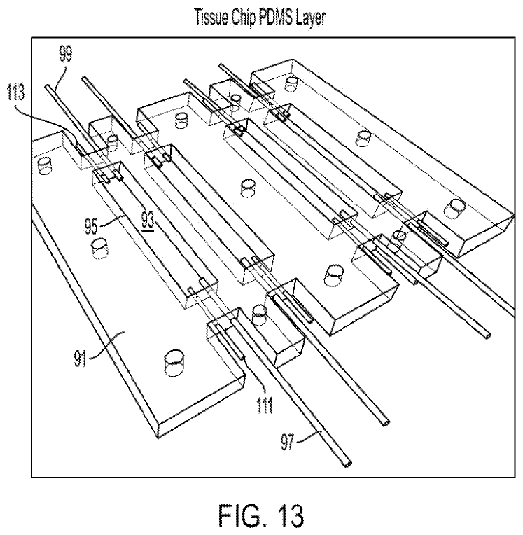

FIG. 13 shows an aspect of a tissue chip that supports culture of a tubular structure while maintaining separation of the luminal and extra-luminal compartments. The aspect shown in FIG. 13 demonstrates a tissue chip, but the general concepts and features described herein can be scaled up or down and used as part of a bioreactor. The tissue chip includes a base 91, an extra-luminal compartment 93, a compartment wall 95, a first hollow cannula 97, a second hollow cannula 99. The extra-luminal compartment 93 is configured to contain a fluid. In some aspects, the extra-luminal compartment 93 can take the form of an open bath, whereas in some aspects the extra-luminal compartment 93 is sealed with, for example, a releasable cover.

Hollow cannulas 97, 99 provide a path between a fluid source and the luminal compartment of the tubular structure 100 (see also FIG. 14). Cannulas 97, 99 can be formed as part of the base 91 or compartment wall 95, or they can be a physically separate material extending through the base 91 or the compartment wall 95. For example, the cannulas 97, 99 can be glass tubes extending through a compartment wall 95 that is formed of PDMS. In some examples, the cannulas 97, 99 may extend down from the top of a bath-style extra-luminal compartment 93. The first and second cannulas 97, 99 extend into the extra-luminal compartment 93 in the depicted aspects, but in other aspects, they may be receded within compartment wall 95. In an alternative construction, a tubular structure 100 with rigid ends 102, 104 might have its ends nested into a channel in the compartment wall 95, negating the need for cannulas to connect the luminal compartment with the fluid source. Note that tubular structures are not coupled to the cannulas and are not shown in FIG. 13.

FIG. 14 shows a magnified view of an extra-luminal compartment, where a tubular structure 100 is fluidically coupled to a first cannula 97 and a second cannula 99. In practice, the extra-luminal compartment will contain a fluid and tubular structure 100 will be at least partially submerged in the fluid. Sutures 109 attach first end 102 of tubular structure 100 to first cannula 97 and second end 104 of tubular structure 100 to second cannula 99. In other aspects, sutures may not be necessary, as the structures may be pressure fit over the cannulas 97, 99, or other forms of support and attachment can be used. In some aspects, for example, supports may be provided as sutures or as features of the base 91 to prevent drooping in one or more central regions of the tubular structure 100.

FIG. 15 shows a fully assembled tissue chip with cover 101 (made of, for example, glass) sealed (by, for example, a compression fit) over the open faced chip in a frame 103 (made of, for example, milled aluminum). In this aspect the fluid column height is used to control hydrostatic pressure differentials between luminal and extra-luminal compartments from the cannula on one end, while fluid pumps attached to cannula at the opposite end regulate fluid flow: Specifically, the extra-luminal compartment 93 (such as is shown in FIG. 13) is fluidically coupled to a first fluid source 105 and the luminal compartment (the interior of the tubular structure 100 shown in FIG. 14) is fluidically coupled to a second fluid source 107. The second fluid source 107 in the depicted aspect includes dye to demonstrate the patency of the wall of tubular structure 100. Fluid from the first fluid source 105 travels into the tissue chip via inlet 111 (shown in FIG. 13), through extra-luminal compartment 93, and out through outlet 113. Fluid from second fluid source 107 flows into first cannula 97, through the luminal compartment of tubular structure 100, and out through the second cannula 99.

With the basic structure of the tubular organoids and the apparatus for making tubular structures being disclosed, a greater appreciation of the construction and benefits may be gained from the following discussion of the operation of the apparatus for making the tubular structures, and the operation of the tubular organoids themselves. It is to be noted that this discussion is provided for illustrative purposes only.

Tubular structures or tubular organoids (tubular structures containing cells) can be formed using the apparatuses shown in FIGS. 3-12 and described above. References to the apparatus will be made to FIG. 3 for demonstration purposes, only, but it is to be understood that the methods of making tubular structures are not limited to the use of the aspect of FIG. 3.

The methods of making tubular structures include coextruding a a luminal solution and one or more wall solutions, with or without an outer carrier solution. The wall solution(s) can be coextruded through outer nozzle 33 (or nested wall solution nozzles in other aspects) and the luminal solution can be coextruded through the inner nozzle 31 positioned coaxially inside the outer nozzle 33. The inner and outer nozzles 31, 33 direct the wall solution and luminal solution into a column-shaped container 35 that is filled with extrusion buffer 41. In this aspect, the wall and luminal solutions are polymerized within the extrusion buffer 41 as they sink to the bottom of the container 35. Once polymerized, the wall solution becomes tubular wall 3 shown in FIG. 2. The container 35 containing the tubular structures 34 can be removed from the housing 21 for collection.

The tubular wall 3 can be extruded to a thickness t of the size ranges described above, and with a thickness t that is fairly consistent along the length of the tubular structure or organoid. The wall solution can be an unpolymerized hydrogel precursor solution of any of the hydrogel wall materials described above. For example, the hydrogel wall solution can be unpolymerized precursors to gelatin methacryloyl (gelma) or PEGDA and may contain additives in one or more nested lavers as described above. The luminal solution can be any of the luminal materials described above. The wall solution, the luminal solution, or both can carry a cell population during extrusion. The cell population can be any of the cell types described above.

The step of polymerizing the wall solution can include photo-crosslinking the wall solution as it sinks within the extrusion buffer 41. One or more light sources, such as light arrays 39, illuminate the wall solution as it sinks within container 35. The wall solution can include photo-initiator components, as discussed above. The light rays 37 are transmitted through the walls of the container 35, which are made of a light-permeable material. The light rays can be UVA, violet, or blue light, in some aspects. In some aspects, the methods can include varying the level of illumination or varying the light wavelength at different points along the length of container 35. For example, in one aspect, the light may be brighter near the top of the container than near the bottom of the container. Varying the light intensity or wavelength at a single point or along length of container 35 can be used to control the rate of polymerization of the wall solution.

The methods of making tubular structures advantageously facilitate the extrusion of long tubular structures. The methods disclosed herein can be used to extrude tubular structures or tubular organoids of at least 10 meters from a first end to a second end of the tubular structure or tubular organoid (including at least 10 meters, at least 9.5 meters, at least 9 meters, at least 8.5 meters, at least 8 meters, at least 7.5 meters, at least 7 meters, at least 6.5 meters, at least 6 meters, at least 5.5 meters, at least 5 meters, at least 4.5 meters, at least 4 meters, at least 3.5 meters, at least 3 meters, at least 2.5 meters, at least 2 meters, at least 1.5 meters, at least 1 meter, at least 0.5 meters, at least 0.25 meters, at least 0.1 meters, and at least 0.01 meters). The long tubular structures or tubular organoids can be used in therapeutic, diagnostic, and/or research applications at the originally extruded length, or they can be cut shorter in further processing steps.

The wall solution can be extruded at a flow rate of about 1 microliter/minute to about 100 microliter/minutes, or more. In some aspects, the wall solution can be extruded at a flow rate from about 1 microliter/minute to about 20 microliters/minute. The luminal solution can be extruded at a flow rate of about 1 microliter/minute to about 100 microliters/minute, or more. In some aspects, the luminal solution can be extruded at a flow rate from about 3 microliters/minute to about 60 microliters/minute. The relative flow rates, in combination with extrusion tip configuration, will help to determine the extruded tube wall thicknesses. Typical inner/outer flow ratios may be for example 1:1, between 4:1 and 1:4, between 1:10 and 10:1 to obtain different wall and lumen thicknesses. The velocity of the fluids can be matched at the point of nested tip extrusion points to minimize turbulent flow and maximize tube uniformity. The relative area ratios in coaxial tip designs can be designed such that at the point of extrusion of upper nested tips into lower nested tips, the velocities can be balanced for a given application of tubule sizes and thicknesses, and thus this aspect of the tip design will contribute to the balance of wall thicknesses and minimizing turbulent flow.

The methods of making tubular structures can further include controlling an environmental condition to optimize a polymerization rate, to optimize cell viability, or both. For example, the environmental regulation system 47 can control one or more variables such as light, temperature, gas levels, and/or humidity, setting the variable to a fixed value or to a particular program of values that may change over time. Controlling the light can include controlling the light intensity, wavelength, or spread angle of the light emitted from light arrays 39. Controlling the light can include turning the lights 39 on and off at particular times. Controlling the temperature can include activating heating and cooling elements inside or connected to the housing 21 to achieve a desired temperature in response to temperature measurements from a thermometer inside housing 21. Controlling the humidity can include releasing water vapor from a water tank inside or fluidically connected to the housing 21 in response to humidity measurements from a humidity sensor inside housing 21. Controlling the gas levels can include releasing gas (oxygen, carbon dioxide, and/or nitrogen, for example) from gas tanks fluidically connected to the housing in response to measurements from gas concentration sensors inside housing 21.

In some aspects, a user can manually control the flow rates and environmental conditions. In some aspects, the user can set flow rates or environmental conditions at a user interface in communication with the environmental regulation system 47. The user interface can be part of a computing device 49 or it can be independent of a computing device. In some aspects, the user interface is directly attached to the housing 21.

The tubular organoids 1 can be used in a variety of therapeutic, diagnostic, and research applications, some of which are disclosed herein. The uses described herein are not intended to be limiting.

In one aspect, the tubular organoids 1 can be used in cytotoxicity testing, where the tubular organoid is contacted with a potential toxin and cellular responses are evaluated.

In one aspect, the tubular organoid mimics a particular organ and is used in an organ function testing protocol. The tubular organoid can be formed using the processes described above, and potentially subjected to a maturation protocol. The tubular organoid is contacted with a test agent or condition (before, after, or during the maturation protocol), and the effects of the test agent or condition on a measure of organ function are monitored. The test agent or condition can be anything that might cause a beneficial or adverse response from the organ, the tubular organoid, or the individual cells of the tubular organoid. The test agent or conditions can include, but are not limited to, potentially therapeutic agents, potentially cytotoxic agents, potentially infectious agents, potentially proliferative agents, mechanical stimuli, variations in temperature, extracellular matrix stiffness, compressive forces, hydrostatic pressure differentials between luminal and ad-luminal compartments, luminal flow rates, luminal sheer, etc.

In another aspect, the tubular organoids can be used to test reproductive therapies (reproductive stimulants or contraceptives). These methods can include forming a tubular organoid 1 that mimics a reproductive structure (such as a testicular structure like, for example, the seminiferous epithelium). The method of forming the tubular organoid can include making a tubular organoid by coaxial extrusion using the methods described above, then subjecting the tubular organoid to a maturation protocol.

The methods can further include contacting the tubular organoid 1 with an agent or condition (before, after, or during the maturation protocol), and monitoring the effects of the agent or condition on a measure of the function of the reproductive structure. In some aspects, the measure of the function of the reproductive structure is a quantity or property (size, ability to form inter-cellular connections and generate a barrier function, for example) of Sertoli cells formed by the tubular organoid. In some aspects, the measure of the function of the reproductive structure is a quantity or property (size, number, proliferative capacity, ability to secrete factors such as androgens) of Leydig cells formed by the tubular organoid. In some aspects, the measure of the function of the reproductive structure is a quantity or property (size, number of chromosomes, or motility, for example) of germ cells formed by the tubular organoid. In some aspects, the measure of the function of the reproductive structure is a quantity or property (size, number, proliferative capacity, ability to secrete factors, ability to contract) of peritubular myoid cells formed by the tubular organoid. In some aspects, the measure of function of the reproductive structure is the tortuosity of the tubular organoid (such as the number or density of coils in a testicular structure, and/or the cellular arrangement maintained in the testicular structure.)

In some aspects, the agent or condition is introduced into the lumen of the tubular organoid. In some aspects, the agent or condition is incorporated into the wall solution or luminal solution before extrusion. In some aspects, the agent or condition is applied to the tubular organoid after extrusion and before, during or after the maturation protocol.

The disclosed tubular organoids 1 can also be used in methods of preserving the fertility of a subject. The methods can include harvesting one or more cells (such as adult, progenitor, or stem cells, or induced pluripotent stem cells, or induced pluripotent derived differentiated cells, or cell lines) from a subject and making a tubular organoid (such as a testicle or testicular structure, for example) with the cell or cells from the subject. The cell from the subject can be carried by the wall solution, the luminal solution, or both. One or more cell types can be incorporated into the tubular organoid. Not all cell types must be from the same subject or source. In some aspects, one cell type may be from the subject, and another cell type included in the tubular organoid can be autologously or xenogenically derived.

The tubular structures described herein can be utilized with tissue chips or bioreactors, such as the tissue chip shown in FIGS. 13-15. Methods of using the tissue chips or bioreactors can include steps of: fluidically coupling an inlet 111 of a tissue chip to a first fluid source 105, fluidically coupling a first end portion 102 of a tubular structure 100 to a first cannula 97, fluidically coupling a second end portion 104 to a second cannula 99, and fluidically coupling the first cannula 97 to a second fluid source 107. The methods further include initiating flow from the first fluid source 105 through the inlet 111 and into the extra-luminal compartment 93, initiating flow from the second fluid source 107 into a luminal compartment of the tubular structure 100, and maintaining patency of a wall of the tubular structure 100 such that the extra-luminal compartment 93 remains separate from the luminal compartment (the interior lumen of the tubular structure 100). The tubular structure 100 can be suspended within the extra-luminal compartment 93 and at least partially submerged in the first fluid that is contained by the extra-luminal compartment 93.

The methods of using the tissue chips or bioreactors can include regulating one or more hydrostatic or hydrodynamic properties of the luminal compartment, the extra luminal compartment, or both. The one or more hydrostatic or hydrodynamic properties can include (but are not limited to), hydrostatic pressure, hydrostatic pressure differentials between the extra-luminal compartment and the luminal compartment, fluid flow rates, shear stresses, and media exchange rates. The regulation of hydrostatic or hydrodynamic properties can be used to exert temporal control over an addition of a factor to a cell culture media (either within the extra-luminal compartment 93, the luminal compartment of tubular structure 100, or both). The hydrostatic or hydrodynamic properties can be regulated using pumps, a gravity feed, a rocking device, a rotating device, fluid restricting channels, and buffer/media wells, for example. In some aspects, a cell culture media factor can be maintained at a different concentration in the extra-luminal compartment than in the luminal compartment.

Some aspects of the methods of using the tissue chip include sampling media from the extra-luminal compartment 93, the luminal compartment, or both. The sampling tests can include an analysis of cells sloughed from the tubular structure 100. In some methods, the analysis may include testing for germ cell maturity. The sampling of media can include an analysis of cellular materials, which include, but are not limited to metabolites, small molecules, proteins, nucleic acids, signaling factors, germ cells, and vesicles. In some aspects, the methods of using the tissue chip include conducting tissue tests on the tubular structure 100. The tissue tests can include, for example, trans-epithelial electrical resistance (TEER) measurements.

In some aspects, the tissue chip or bioreactor can include a cover 101 that can be releasably sealed to base 91 of the tissue chip to provide temporary access to the extra-luminal compartment 93. This can facilitate addition or removal of tubular structures 100 and can provide access for tissue testing and media sampling. However, a releasable cover may not be necessary to conduct these tests or to sample media, and its description herein is not intended to limit the disclosure.

Once coextruded or seeded with cells following generation of the extruded tubular structure, a tubular organoid can be subjected to a maturation protocol. In some aspects, the maturation protocol can be performed within a bioreactor or a tissue chip, such as the tissue chip shown in FIGS. 13-15. For example, a tubular organoid can be subjected to a testicular maturation protocol that induces the tubular organoid to mimic a mature testicular structure (such as the seminiferous epithelium). The tubular organoid can then be preserved for an extended duration (cryogenically, for example, or maintained in longer term cultures). At a later time-point, the tubular organoid can be used to perform a desired organ function. For example, a cryogenically preserved testicular structure can be thawed and can be used to produce haploid germ cells, which can be harvested from the testicular structure and used for fertility treatments.

### EXAMPLE

The working system described in this Example prints tubular structures or organoids relevant to developing, pubertal, and adult seminiferous epithelium in various species (˜50-300 microns). The tubular structures can be seeded with testicular cells to form tubular organoids. Over time, under certain conditions, the tubular organoids coil as would be expected in testicular development. In vitro spermatogenesis can be investigated with this system, as can general epithelial-mesenchymal interactions for coiled tubular tissue generation, and the contributions of different testicular cell types to seminiferous tubule functions and spermatogenesis. The system can further be used to test effects of mechanical stimuli on biological responses (matrix stiffness/flexibility, hydrostatic pressure differentials between luminal and ad luminal compartments, effect of hydrodynamic forces such as flow rate/sheer in luminal and ad luminal compartments, etc.). The system can also be used to investigate the role of matrix vs, smooth muscle cells in tubular tissue coiling, which is applicable to the development of several tissues. The system can further be used to investigate the effects of tube size and wall thickness on mechanical properties and nutrient/gas exchange in several areas of tissue engineering. The aspects of the tubular constructs can be used to investigate material properties such as stiffness, porosity, surface texture, permeability, and effects of additive materials in bioengineering tubular tissues.

The insulated housing of the apparatus can be formed, for example, of acrylic or steel. An upper compartment houses syringe pumps and is protected from UV so that raw material is not polymerized. The extrusion nozzle is situated between the upper and lower compartments in a holder that can accommodate tips with differing configurations but with the same outer seating frame so that they are exchangeable within the device. The lower compartment houses the LED arrays configured for consistent exposure and the UV transmissible tubes, such as quartz tubes, that hold buffer for extrusion. Custom designed quartz tubes and extrusion nozzles are assembled in the chassis. High intensity LEDs are commercially available with blue to long wave-length UV (and the optimal light spread angles for curing). These LEDs can be assembled with controllers for tunability, and can be incorporated into arrays that optimize for consistent light exposure. Consistent controlled light exposure over time after extrusion helps to set tube structure while minimally exposing cells. The light ballasts, regulators, and heat components are housed in the rear of the unit and connected to control panels on the front. The upper chamber is accessed from above via an access door (for hydrogel and cell loading). The bottom panel is accessed by front access door for loading/exchange of the quartz tubes where tube gelation is achieved.

In some configurations, the light array has 1-3 rows of LEDs positioned with consideration of the light spread angle. In some designs, some or all of these light arrays are on a swivel so that they can be appropriately positioned once the collection tube is in place. In some designs, such as if one or two rows of LEDs are sufficient, then the swivel feature will not be needed. In some designs there is a single source of light for photo-curing.

Apparatus examples: Additional variations and aspects of the apparatus for making tubular structures are shown in FIGS. 10-12. FIG. 10A shows a side view of a full apparatus 200. FIG. 10B shows a side view of the bottom portion 204 of the apparatus 200. FIG. 10C shows a top-down view of the example apparatus 200, as though looking from line a-a of FIG. 10A. The example apparatus 200 is on an air table 201. The apparatus 200 includes both a top portion 202, a central portion 203, and a bottom portion 204 surrounded by a static casing 206. The top portion 202 includes syringe pumps which are each coupled to extrusion nozzles 208. The extrusion nozzles discharge a photocrosslinkable polymer into a column-shaped container 210. which extends through central portion 203 and is coupled to the static casing 206 via tube clamps. In the central portion 203, the column-shaped container is surrounded by an LED array 212, which cure the photocrosslinkable polymer as it travels down the column-shaped container. The LED array 212 is mounted on the static casing 206 and includes a constant current driver, a constant voltage power supply, and a dimmer. The syringe pumps and nozzles are in the top portion 202 shielded from the light of the LED array 212. A door 214 is mounted to the static casing 206 via a pivot point 214, which allows loading of the column-shaped container. A portion of the bottom of the column-shaped containers is blocked from the LED array to create a “dark zone” (in bottom portion 204) where the cured polymer can be collected and any included cells can be protected from the light. The bottom portion 204 is mounted on a scissor jack 216 or a similar lift to adjust the height of the container and, thus, the height of the dark zone within the container. The temperature in the column-shaped containers is monitored by a platinum RTD 1000 W temperature sensor and controlled by a derivative or PID controller, which controls a heating system in the rear of the apparatus that expels temperature-conditioned air into the main part of the apparatus via a filtered duct system. The heater can operate at either 230 V or 110 V, have a flow rate of approximately 20-50 CFM, and have a heat duty of approximately 140 W.

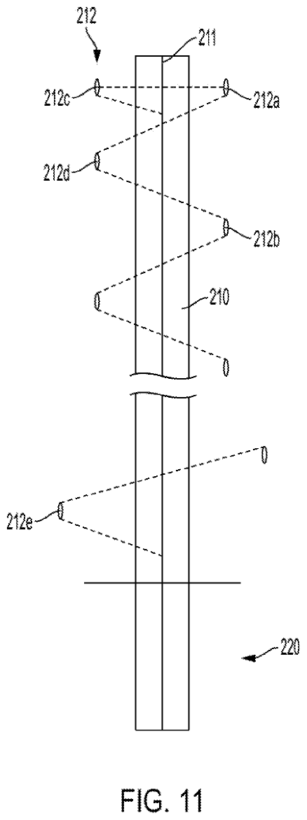

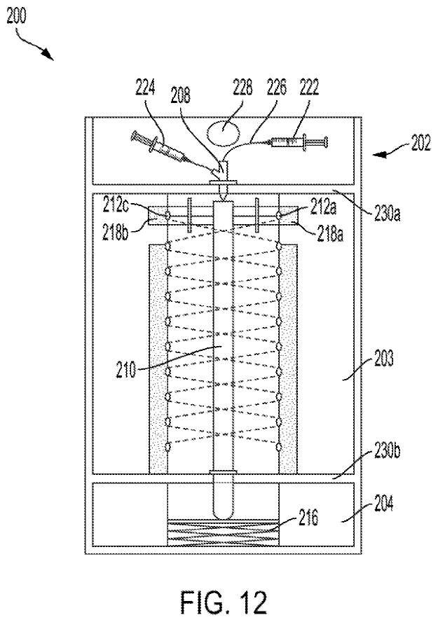

FIG. 11 shows a side view of an exemplary LED array 212 surrounding a container 210. Each LED light 212a has a light spread of 55°, and the LEDs can be angled with different spacing and/or configurations. The horizontal spacing of the individual LEDs will impact the irradiance: the closer an LED is to the center of the tube, the more intense the irradiance. The LEDs in the exemplary array are spaced about 2.5 cm to about 4 cm from the vertical center 211 of the container 210 depending on their vertical position. The vertical spacing of the LEDs will determine the overlap of light spread. The LEDs in the exemplary array are spaced about 2.5 cm apart on alternating sides of the column-shaped container (i.e., two LEDs on the same side 212a, 212b are 5 cm apart). Looking at FIG. 12, the first 2 LEDs 212a, 212c closest to the top of the container 210 can be on a vertical slider 218 to allow their position to be adjusted and angled to avoid irradiating the nozzle. In the exemplary array, these first 2 LEDs 212a, 212c are positioned at the same vertical height on opposing sides of the container 210, which creates a higher irradiance that “sets” the cure of the photocrosslinkable polymer. The remaining LEDs 212b, 212d (and so on) are positioned in an alternating pattern down the remainder of the container 210 to create a lower irradiance as the photocrosslinkable polymer travels down the length of the container 210. The end of the container 210 is a “dark zone” 220 where the cells encapsulated in the photocrosslinkable polymer are protected from prolonged light exposure. If the lowest LED 212e has a 55° beam and is 2.5 cm horizontal from the center of container 210, it could be placed at least 2 cm above the start of the dark zone 220; however, more space (potentially 3 cm or more) may be needed depending on the height of the dark zone 220.

FIG. 12 shows a side view of an example apparatus 200 for making tubular organoids and tubular structures. The top portion 202 of the apparatus has two syringe pumps 222, 224 connected to an extrusion nozzle via Tygon type inert tubing 226, which are all enclosed within the apparatus 200 by an access door 214 (shown in FIG. 10) that can be used to load and unload the syringe pumps. The middle portion of the apparatus includes a portion of a column-shaped container 210 which is surrounded by an LED array. The two LEDs at the top of the array are mounted on sliders 218a. 218b on either side of the column-shaped container: these two LEDs 218a, 218b will be primarily responsible for setting the photocrosslinkable polymer that is extruded through the extrusion nozzles 208. Because the LED array 212 and column-shaped container 10 are narrow; the central portion 203 also fits light ballasts or light controllers, a heater, and a heat controller. The heater is an in-line duct fan with an in-line HEPA filter near the duct output. To avoid overheating, the top portion and the middle portion each have separate duct outputs, but a single duct input 228 is positioned in the top portion 202. The heater controller is a derivative/PID controller. The bottom portion 204 of the apparatus 200 includes the bottom of the column-shaped container mounted on a lift 216, which allows the position of the column-shaped container 210 to be adjusted. The top, middle, and bottom portions are separated by diffusion plates 230a, 230b (e.g., plexiglass with holes to diffuse flow), which blocks light from the LED array from entering the top portion and the bottom portion but helps distribute heat flow.

System components and design: The system developed uses coaxial or multiaxial extrusion of photo-cross linkable material such as gelma or PEGDA to manufacture small diameter tubes for bioengineering applications. Coaxial extrusion of gelma with photo-curation without additional curing agents into buffer using laminar flow extrusion to make these tubes is a novel method. Printing of radially symmetrical gelma tubes of these small diameters is a novel product. The combination of components to make a device to do this is a novel device. The configuration of the extrusion tips is a unique modular concept for coaxial or multiaxial extrusion.

Cells can be seeded into tube walls or into luminal area at extrusion, or seeded in the luminal space with syringe pumps or manual canulation after extrusion in standalone or tissue-chip configurations, and/or seeded on the exterior surface of the tube. Various combinations of cell seeding techniques can be used at different times with the different cells of the testis (Sertoli, germ-line, myoid smooth muscle, Leydig) to recapitulate seminiferous epithelium and testis organoids.

Tip designs started with using plastic pipette tips and blunt needles with different gauge size openings (modified with cutting/grinding with razors, side-cutters. Dremel tool, drills etc to fit into coaxial extrusion tips). Adhesives used to glue the pieces together included cyanoacrylates, E6000, and dental epoxy. To optimize the cleaning and sterilization processes, bulk length needles specialty 38octite adhesives can be used. Alternatively, stainless steel components with luer-lock fittings can also be autoclaved for sterilization.

The device utilizes tips with pore gauge sizes of 50-300 microns, where outer and one or more nested inner extrusion pores are on separate connectable pieces that are self-centering such that combinations in the coaxial extrusion device can be easily switched out. The tips can be cast or milled (for example, precision milled), and/or 3D printed or sintered. 3D printing and/or sintering can also allow for single part tips with all nested tips as a single part. The coaxial extrusion pieces can be modular and threaded (screw thread or luer-lock) with or without a base to seat in the frame of the extrusion device system in a holder for secure seating. The inlets for inner and outer flow tubing connection to syringe pumps can be pegs directly manufactured on the inner tip or ˜17-gauge blunt needle stubs adhered into pores after manufacture. The space between inner and outer tips tapers to aid in reduction of the tubule size and reduce sheer on any cells being co-extruded. Sheer is further reduced and sizes of tubes can be further reduced in multiaxial aspects where the outer material extruded is not polymerized but acts as a carrier. The space within the outer tip, after the inner tip ends, also tapers and then levels to final gauge to stabilize laminar flow. This assists in compressing the tubes down to final size. The ratio of cross-sectional area at the point the inner tip ends is matched to final ratios desired to achieve a particular wall thickness (which is customizable by mixing and matching inner and outer tips, tip area ratios, and material flow rates). Syringe pump relative flow rates are matched to these ratios to match fluid velocities and avoid chaotic flow which is seen when the flow ratios and area ratios differ by too much. The edge of the outer tip is as thin as possible to avoid extruded material from shifting along the edge at extrusion and diverting flow to the side (which has been observed with thicker needle wall thicknesses), especially when the outer material is polymerized such as in a two-tip nested aspect. The point can be manufactured to a coring point (for example, by milling). To stabilize the tips, the wall thickness tapers from the tip to thicker wall for support. Extrusion pores of various shapes are possible-for example, with crypts and/or ridges to mimic oviductal or intestinal epithelial tubes. In these aspects, density matching is advantageous.

During extrusion, the coaxial extrusion tips are connected to syringe pumps with inert tubing, preferably a tubing that allows for gas exchange so that cells in the system will have gas exchange during extrusion (for example, tygon tubing). The syringes used are also preferably gas diffusible. Screw drive syringe pumps facilitate very precise flow control to the sub microliter-minute (for example, Fusion 200, Chemyx Inc.).

Performing the extrusion in a temperature-controlled environment helps to prevent the gelma from gelling and contributes to high cell viability. One option is to perform the extrusion in a cell incubator with gas, temperature and humidity control. Another option is to design the device with an environmental control system that maintains an appropriate temperature range during extrusion. In one aspect, the environmental control system includes a fan driven heat exchanger and an associated control system.

The gelma tubes are extruded into buffer in collection tubes (containers) that are UV transmissible, or from nozzle tips where the outer nozzle is UV transmissible for in-tip curing. For example, the collection tubes can be UV transmissible acrylic square tubes. Or, for example, the collection tubes can be UV transmissible quartz sleeves. One aspect utilizes a quartz sleeve with a wall about 1-2 millimeters thick, about 2 centimeters in inner diameter, and about 12 centimeters long. The diameter will influence keeping the extruded material in the UV curing light path as it sinks and reduces back current from displaced buffer flowing the opposite direction.

Light arrays, such as, for example, LED 395-405 nanometer lights, can be secured parallel to the collection tubes to cure the extruded gelma tubes as they flow out the nozzle and sink in the buffer. In one example, the light array is a C3535U-Unx1 High Power UV LED (SemiLEDS, Inc.). As another example, a dimmable flashlight can be used to cure the gelma tubes.

### General Instructions for Principles of Extrusion and UV Fixing

The relative flow rates during extrusion to get desired results of tube sizes will also depend on the tip design, such as the tip diameter ratios and lengths, and the total flow rates used. As an example, the range of relative flow rates is from about 1:1 to 2:7 when the inner tip diameter is ⅕- 1/10^{th} the outer tip diameter at its point of extrusion into the outer tip, the outer tip is approximately 150 μm, and the total flow rate is between 10-50 μl/min. Gelma concentrations of about 10% work well for tube formation, and a range of 5-15% can be used (with increasing stiffness and decreasing permeability noted in the extruded tube product). Velocity matching can be further considered in balancing flow rates and area ratios to minimize turbulent flow: Photoinitiators which mediate Gelma polymerization upon UV exposure include Lithium acylphosphinate photo-initiator (LAP), which can be used at about 0.5%, but which can be used at differing concentrations to get desired tube properties based on Gelma polymerization. LED UV exposure of the extruded tubes is typically at a higher intensity, such as about 45-90 mW/cm², for a short blast to set the tube, followed by a longer exposure at lower intensity to cure the tube through the center and avoid collapse of the tubular structure (loss of tubular lumens) upon settling at the bottom of the extrusion container.

### Method to Use this System to Generate 3D Tubes

Preliminary preparation for procedure: All supplies are sterilized by appropriate means. For example, the apparatus/device can be sterilized in a gas sterilizer and then stored in a sterile environment such as a laminar flow hood or biosafety cabinet. If housed there, and used under sterile technique conditions, it can then be wiped down with ethanol to clean between uses such that gas sterilization is not required every time. Components used to prepare cells or run cell suspensions should be sterilized before each use (collection tubes, extrusion syringes and tubing, extrusion tips, extrusion/polymerization buffers to fill collection tubes, etc). Alternatively when cells are seeded after tubule extrusion, the tubules can be sterilized after production, for example by irradiation.

Sterile hydrogel precursor is prepared or purchased. For example, sterile, lyophilized gelma precursor powder can be prepared from commercial gelatin. For example, a preparation can be made using 5-15% wt/vol of gelatin dissolved in warmed carbonate buffer, pH 9.0, and functionalized with methacrylic anhydride. Once the reaction is complete, the solution is diluted 1:5 with water and dialyzed. This Gelma precursor can be filter sterilized before lyophilization, then lyophilized in sterile filter top conical tubes and kept sterile thereafter or in non-sterile tubes. The gelma and photoinitiator volumes that will be needed for rehydration are calculated and mixed prior to the procedure and additional additives can be added for each desired layer for the application. In some examples, the final concentrations at polymerization will be 5-15% of ˜80% functionalized gelma, 3% PEGDA, and 0.3-1% Lithium phenyl-2,4,6-trimethylbenzoylphosphinate (LAP) photoinitiator, depending on the stiffness desired in the final product.

In some examples, the hydrogel precursor can be prepared from or with tissue specific extracellular matrix extracts (such as decellularized extracellular matrix, for example). The extracellular matrix extracts can be functionalized to facilitate crosslinking.

Preparation of luminal solution: The luminal solution could be, for example, a sugar syrup, a PVP containing buffer, or a gelatin solution. The density and viscosity of the luminal solution should be similar to that of the hydrogel precursor solution to reduce artifacts that can appear during extrusion due to disrupted flow patterns and to reduce the buoyancy of the extruded tubules. As an example, a 10% w/v gelatin luminal solution is a good density/viscosity match for a 10% w/v gelma wall solution (the solution that ultimately becomes the walls of the tubes). Luminal gelatin solution can be filter sterilized and stored refrigerated for up to 2-3 weeks (though perhaps shorter if specialty cell culture media is used to dissolve the gelatin and support cells). If seeding cells into the luminal compartment at extrusion, they can be resuspended directly in the 10% gelatin solution.

Cell Culture: Cells are cultured and harvest according to cell-specific protocols and in time to be ready on the day of extrusion. Any special cell culture media needed to receive and culture organoids is prepared in advance.

Procedure steps: The device is set up with the appropriate extrusion tip configuration for the desired outcome. Outer tip diameter and/or flow volume of outer tip carrier material determines overall diameter of product. Nested inner tip sizes along with relative flow rates of solutions and inner selected material (gelatin or other) affects the final product, dependent on placement within the nested tips. More specifically, the ratio of flow at the cross-sectional area at the point of the extrusion tip where an upper inner tip ends should match the proportional flow rates on the syringe pumps and in this case will also correlate to the proportion of the extruded tube that is ‘wall’ vs lumen. Consideration of velocity matching when calculating tip specifications will improve the product.

Next, the syringe pump hoses are connected to the assembled co-axial extrusion tip. The co-axial extrusion tip is inserted into the first collection tube with buffer. The syringe pumps are set to appropriate settings (syringe type and flow rate for each). The LED source is set to desired settings according to recommendations and desired outcome. The temperature is set according to the desired outcome (typically between 33-37 degrees Fahrenheit). The unit is closed and ample time is given to allow for temperature to equilibrate.

Next, fresh wall solution(s) (gelma, for example) is prepared. Wall, luminal, and carrier (if used) solutions are equilibrated to the desired temperature. Cell preparations for co-extrusion are suspended as single cell suspensions at high density. After dilution into the wall solution(s), the final density can be between about 6 and about 30 million cells/ml depending on cell type proliferative capacity and the final organoid product desired.

The cell preparations are mixed into the wall or the luminal solution according to the organoid production goals at around 33-37 degrees by drawing cells into syringe with the solution and either leaving a large bubble to facilitate mixing by inversion, or by using a syringe coupler to mix, then burp, the syringe, loading into the syringe pump. Next, the extrusion device is primed, each layered solution, to remove all air bubbles. Then the syringe pumps are started and allowed to flow until the coaxial tip is primed, free of bubbles, and exhibits desired flow. The extrusion, polymerization, and tube collection continues until the desired tube length is collected. For comparing tubes produced under different flow rate combinations, containers should be changed in between (to keep track of samples). If altering variables requires changing inner or outer tips of the coaxial system, the tips should be burped and primed again.

To prevent contamination of the exterior of the organoid with cells intended for the luminal compartment: The luminal solution will not polymerize and or solidify, so if cells are mixed therein, they may leak from the ends of the tube into the container and potentially adhere to the exterior surfaces of the tubular organoids. To prevent this, a layer of polymerizing material can be run by itself, before any free cell solutions such as non-polymerizing luminal materials. Then, the collection tubes are switched. The light source is immediately turned on to cure the wall solution and create a sealed end to the tubular organoid. Then luminal flow is restarted. When the end of the desired extrusion is drawing near, luminal flow is stopped again and only wall solution is allowed to flow, which seals the back end of the tube. This will prevent leaking of cells from the luminal compartments into the bottom of the collection vessel where they would adhere to extruded tube exteriors.

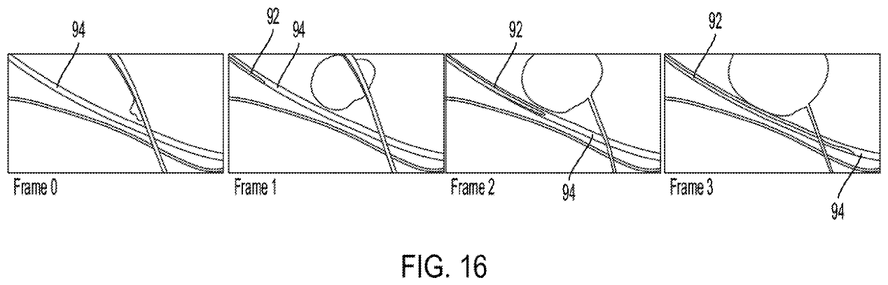

Patency of tubular constructs: The walls of the tubular constructs were fully patent, with no leaks. FIG. 16 shows stills from a video where an extruded tubular structure was manually cannulated with a 34-gauge needle and dye 92 injected into the tubular construct 94. Each frame shows the advance of the dye within the formed tube, demonstrating that the construct was hollow and consistently constructed.

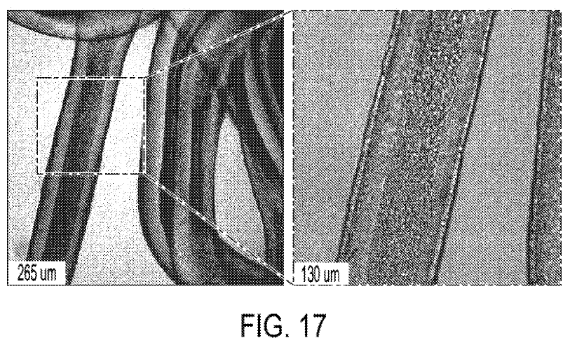

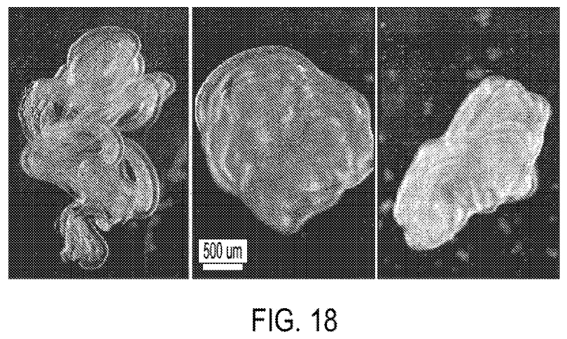

Tubular constructs seeded with seminiferous epithelial cells: This system has been used to generate tubular constructs seeded with cells of the seminiferous epithelium of the testis (Sertoli cells) (FIG. 17). The tubular constructs were cultured to allow proliferation and development of organoids. The constructs demonstrated coiling as the epithelial cells proliferated and colonized over a week in culture (FIG. 18). Interestingly, this particular model can be used to address questions on the distinct roles of epithelial cells and smooth muscle cells in tissue coiling. Because the model of FIGS. 17-18 does not incorporate smooth muscle cells, it can be used to explore epithelial cell mediated tissue coiling based solely on matrix properties rather than smooth muscle signaling mechanisms.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The implementation was chosen and described in order to best explain the principles of the invention and the practical application, and to enable others of ordinary skill in the art to understand the invention for various implementations with various modifications as are suited to the particular use contemplated.

### REFERENCES

Wang et al., Coaxial extrusion of tubular tissue constructs using a gelatin/GelMA blend bioink, ACS Biomater. Sci. Eng. 2019, 5, 10, 5514-5524.

1. A tubular structure comprising:
a tubular wall including a hydrogel material, the tubular wall comprising a first end, a second end, and a radial thickness ranging from 1 micron to 200 microns;
a lumen extending through the tubular wall and comprising a luminal diameter defined by inner surfaces of the tubular wall; and
a luminal material within the lumen.

2. The tubular structure of claim 1, further comprising a cell population living in the luminal material, in the tubular wall, or both.

3. The tubular structure of claim 2, wherein the cell population comprises epithelial cells.

4. The tubular structure of claim 1, wherein the cell population comprises smooth muscle cells.

5. The tubular structure of claim 1, wherein the hydrogel material is free of divalent cations.

6. The tubular structure of claim 1, wherein the hydrogel material is free of alginate.

7. The tubular structure of claim 1, wherein the hydrogel material comprises gelatin methacryloyl.

8. The tubular structure of claim 1, wherein the hydrogel material comprises decellularized extracellular matrix.

9. The tubular structure of claim 1, wherein the hydrogel material comprises an additive configured to alter at least one material property of the formed hydrogel.

10. The tubular structure of claim 9, wherein the at least one material property comprises a strength, a porosity, a stiffness, a surface texture, and a cell adhesiveness.

11. The tubular structure of claim 1, wherein the luminal material comprises gelatin.

12. The tubular structure of claim 1, wherein an outer diameter of the tubular wall ranges from 5 microns to 300 microns.

13. The tubular structure of claim 12, wherein a standard error of the outer diameter is from 3% to 10% of the outer diameter.

14. The tubular structure of claim 1, wherein a standard error of the radial thickness of the tubular wall is from 5% to 20% of the radial thickness.

15. The tubular structure of claim 1, wherein the tubular wall has a plurality of radial wall layers.

16. The tubular structure of claim 1, wherein the luminal diameter ranges from 1 micron to 200 microns.

17. The tubular structure of claim 16, wherein a standard error of the luminal diameter is less than 8%.

18. The tubular structure of claim 2, wherein the cell population comprises testicular cells.

19. The tubular structure of claim 18, wherein the tubular structure mimics a seminiferous tubule.

20. The tubular structure of claim 18, wherein the tubular structure produces haploid germ cells.

21-92. (canceled)
